(19) United States
(12) Patent Application Publication
(10) Pub. No.: US 2023/0347123 A1
(43) Pub. Date: Nov. 2, 2023

(54) FLUID DELIVERY APPARATUS WITH MICRONEEDLES

(71) Applicant: Sorrento Therapeutics, Inc., San Diego, CA (US)

(72) Inventor: Russell Frederick Ross, San Diego, CA (US)

(21) Appl. No.: 17/784,994

(22) PCT Filed: Dec. 2, 2020
(86) PCT No.: PCT/US2020/062863

§ 371 Date: Jun. 13, 2022

Related U.S. Application Data
(60) Provisional application No. 62/942,971, filed on Dec. 3, 2019.

(51) Int. Cl. A61M 37/00; A61M 5/168

(52) U.S. Cl. CPC A61M 37/0015; A61M 5/16804; A61M 2037/0023

(57) ABSTRACT

Disclosed herein are devices and methods for delivering a fluidic composition across a dermal barrier of a subject, e.g., into the lymphatic vasculature of the subject. In some embodiments, the devices and methods provide improved and/or uniform flow rates through a plurality of protrusions defined on the base of a fluid distribution assembly of the device. In some embodiments, the device comprises an attachment band assembly having an annular body comprising a wall defining a hollow inner space and a coupling member to engage with a corresponding coupling member of a collet, and a strap assembly removably engaged with the annular body

### CROSS-REFERENCE TO RELATED APPLICATIONS

This application claims priority to U.S. Provisional Patent Application No. 62/942,971 filed Dec. 3, 2019 and entitled “IMPROVED FLUID DELIVERY APPARATUS,” the contents of which is incorporated by reference herein in its entirety.

### DESCRIPTION

### Introduction and Summary

The present disclosure relates generally to a fluid delivery apparatus, and more specifically relates to an injectable fluid delivery apparatus. The present disclosure also relates to methods of applying a fluid delivery apparatus to a subject's skin to deliver a fluidic composition across a dermal barrier of a subject.

Conventionally delivery forms for small-molecule drugs and biological agents in various clinical applications include subcutaneous injections, intravenous infusions, oral tablets, nasal sprays, but these methods present difficulties. Intradermal or subcutaneous administration can be painful. A large first pass effect is seen with oral administration, which causes delayed onset of therapeutic effects. These methods also are not suitable for treatment of diseases of the lymphatics, which requires administration of drugs directly into the lymphatics.

There is a need for intra-lymphatic drug delivery to provide improved efficacy through more effective concentrations in the disease areas, lymphatic system, and lymph nodes, and/or through achieving biological or clinical effects for drugs active in the lymphatics with reduced dose levels. Such intra-lymphatic drug delivery can provide advantages over other methods of drug delivery, including achievement of systemic exposures faster than oral delivery, avoiding the first pass effect, and extended PK profiles for drugs that have a half-life when administered by other routes.

In one aspect, the present disclosure provides an injectable fluidic delivery device to improve the efficacy and safety of small molecules and biological agents through tunable pharmacokinetics (PK) and intra-lymphatic drug delivery. The device includes an array of active-hollow micro-sized protrusions covered with a nanopatterned layer with a fluidic distribution assembly that can precisely control the flow out of each protrusion. After device activation, the protrusions penetrate the skin to a depth that is distributed between the epidermal and dermal skin layers proximal to the initial lymphatic capillaries. This location of the protrusions can create a predominately unidirectional mass transfer towards the initial lymphatic capillaries. In comparison, conventional subcutaneous injection results in a multidirectional mass transfer that diffuses through Brownian motion in all directions and reduces drug delivery to the initial lymphatic capillaries. In addition, the nanopatterned layer that covers the protrusions can further enhance intra-lymphatic drug delivery through increased paracellular and transcellular transport through the epidermal and dermal skin layers.

The transport properties and positioning of the protrusions in the skin can facilitate tunable PK profiles and increased intra-lymphatic delivery versus traditional routes of drug administration. The array of protrusions can also make the device less painful and more comfortable for patients compared to other forms of drug administration and could facilitate at-home treatments. Injectable fluidic delivery devices according to the present disclosure also provide a collet and body attachment system for maintaining consistent penetration depth in the skin until the administration is complete.

Accordingly, the following embodiments are provided.
Embodiment 1. A device for delivering a fluidic composition across a dermal barrier of a subject, the device comprising:
   a. a fluid distribution assembly comprising:
      i. a base;
      ii. a plurality of protrusions defined on the base, wherein each of the protrusions has a tip and a height at a microscale with a fluidic path defined therein along the height from the base;
      iii. a nanopatterned layer comprising a plurality of nanostructures and covering a surface of the plurality of protrusions;
      iv. a gasket comprising a pressure-sensitive adhesive (PSA) layer;
      v. a fluidic distribution manifold configured to be fluidically connected with the fluidic path of the protrusions and to controllably distribute the fluidic composition among the plurality of protrusions through the fluidic path;
   b. a plenum assembly slidably coupled to the fluidic block and configured to hold the fluidic distribution assembly;
   c. a collet assembly constituting the housing of the device and configured to contact a surface of the subject's skin sufficient for penetration of the plurality of protrusions into the surface of the subject's skin and across the dermal barrier; and
   d. a controller assembly slidably coupled to the fluidic block and configured to control the flow of the fluidic composition during delivery of the fluidic composition through the plurality of protrusions;
wherein the number of protrusions in the plurality of protrusions is from about 4 to about 3000 protrusions and the device is capable of controllably delivering the fluidic composition to a location below the dermal barrier at a flow rate of greater than about 0.1 μl/hour per protrusion, or at a flow rate ranging from about 0.1 μl/hour to about 10 μl/hour per protrusion.
Embodiment 2. A device for delivering a fluidic composition across a dermal barrier of a subject, the device comprising:
   a. a fluid distribution assembly comprising:
      i. a base;
      ii. a plurality of protrusions defined on the base, wherein each of the protrusions has a tip and a height at a microscale with a fluidic path defined therein along the height from the base;
      iii. a nanopatterned layer comprising a plurality of nanostructures and covering a surface of the plurality of protrusions;
      iv. a gasket comprising a pressure-sensitive adhesive (PSA) layer;
      v. a fluidic distribution manifold configured to be fluidically connected with the fluidic path of the protrusions and to controllably distribute the fluidic composition among the plurality of protrusions through the fluidic path, the fluid distribution manifold comprising:
         an inlet channel;
         a plurality of supply channels and resistance channels, wherein each supply channel is connected to a respective resistance channel that facilitates an increase in the resistance to the flow of the fluid;
         an outlet channel aligned with and fluidically connected to the fluidic path of the protrusions;
   b. a plenum assembly slidably coupled to the fluidic block and configured to hold the fluidic distribution assembly;
   c. a collet assembly constituting the housing of the device and configured to contact a surface of the subject's skin sufficient for penetration of the plurality of protrusions into the surface of the subject's skin and across the dermal barrier; and
   d. a controller assembly slidably coupled to the fluidic block and configured to control the flow of the fluidic composition during delivery of the fluidic composition through the plurality of protrusions;
wherein the device is capable of delivering the fluidic composition at a flow rate greater than about 0.1 μl/hour per protrusion, or at a flow rate ranging from about 0.1 μl/hour to about 10 μl/hour per protrusion.
Embodiment 3. A device for delivering a fluidic composition across a dermal barrier of a subject, the device comprising:
   a. a fluid distribution assembly comprising:
      i. a base;
      ii. a plurality of protrusions defined on the base, wherein each of the protrusions has a tip and a height at a microscale with a fluidic path defined therein along the height from the base;
      iii. a nanopatterned layer comprising a plurality of nanostructures and covering a surface of the plurality of protrusions;
      iv. a gasket comprising a pressure-sensitive adhesive (PSA) layer;
      v. a fluidic distribution manifold configured to be fluidically connected with the fluidic path of the protrusions and to controllably distribute the fluidic composition among the plurality of protrusions through the fluidic path;
   b. a plenum assembly slidably coupled to the fluidic block and configured to hold the fluidic distribution assembly;
   c. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
   d. a controller assembly slidably coupled to the fluidic block and configured to control the flow of the fluidic composition during delivery of the fluidic composition through the plurality of protrusions;
   e. an attachment band assembly configured to couple to the collet assembly to facilitate contact with a surface of the subject's skin sufficient for penetration of the plurality of protrusions into the surface of the subject's skin and across the dermal barrier, wherein the attachment band assembly comprises:
      an annular body comprising a wall defining a hollow inner space and a coupling member to engage with a corresponding coupling member of the collet, wherein the annular body is configured to attach to the collet of the collet assembly; and
      a strap assembly removably engaged with the annular body and comprising a hoop-and-loop type fastening strap such that in use, the strap is threaded through a portion of the annular body and folded back to tighten the strap around the skin of the subject.
Embodiment 4. A device for delivering a fluidic composition across a dermal barrier of a subject, the device comprising:
   a. a fluid distribution assembly comprising:
      i. a base;
      ii. a plurality of protrusions defined on the base, wherein each of the protrusions has a tip and a height at a microscale with a fluidic path defined therein along the height from the base;
      iii. a nanopatterned layer comprising a plurality of nanostructures and covering a surface of the plurality of protrusions;
      iv. a gasket comprising a pressure-sensitive adhesive (PSA) layer;
      v. a fluidic block configured to be fluidically connected with the fluidic path of the protrusions and to controllably distribute the fluidic composition among the plurality of protrusions through the fluidic path;
   b. a plenum assembly slidably coupled to the fluidic block and configured to hold the fluidic distribution assembly, comprising
      a plenum having a center portion removably connected to a tubing system to provide a pressured flow of the fluidic composition from a reservoir for holding the fluidic composition located exterior of the device; and
      a plenum cap assembly configured to facilitate gas extraction from the fluid;
   c. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
   d. an external infusion pump configured to control the flow of the fluidic composition during delivery of the fluidic composition through the plurality of protrusions;
   wherein the device is capable of controllably delivering the fluidic composition to a location below the dermal barrier, at a flow rate greater than about 0.1 μl/hour per protrusion, or at a flow rate ranging from about 0.1 μl/hour to about 10 μl/hour per protrusion.
Embodiment 5. A device for delivering a fluidic composition across a dermal barrier of a subject, the device comprising:
   a. a fluid distribution assembly comprising:
      i. a base;
      ii. a plurality of protrusions defined on the base, wherein each of the protrusions has a tip and a height at a microscale with a fluidic path defined therein along the height from the base;
      iii. a nanopatterned layer comprising a plurality of nanostructures and covering a surface of the plurality of protrusions;
      iv. a gasket comprising a pressure-sensitive adhesive (PSA) layer;
      v. a fluidic block configured to be fluidically connected with the fluidic path of the protrusions and to controllably distribute the fluidic composition among the plurality of protrusions through the fluidic path;
   b. a plenum assembly slidably coupled to the fluidic block and configured to hold the fluidic distribution assembly, comprising
      a plenum component;
      a cannula around a center axis coupled in fluid communication with the fluidic block; and
      a plenum cap assembly configured to facilitate gas extraction from the fluid;
   c. a cartridge assembly comprising a reservoir component includes an upper cavity and an opposing lower cavity coupled together in flow communication with the fluidic block via the cannula of the plenum assembly;
   d. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
   e. a controller assembly slidably coupled to the fluidic block and configured to control the flow of the fluidic composition during delivery of the fluidic composition through the plurality of protrusions, comprising:
      a plunger member positionable in a range from a first position proximal to the plenum, to a second position distant from the plenum; and
      a bias assembly positioned between the plenum and the plunger member, the bias assembly being configured to apply a pressure to the plunger member, wherein the pressure applied to the plunger member by the biasing assembly is transmitted to the plenum and facilitates displacing the fluidic composition into the fluidic block.
   wherein the device is capable of penetrating the dermal barrier of the subject and controllably delivering the fluidic composition to a location below the dermal barrier, at a flow rate greater than about 0.1 μl/hour per protrusion, or at a flow rate ranging from about 0.1 μl/hour to about 10 μl/hour per protrusion.
Embodiment 6. The device of any one of claims 1-5, wherein the device is capable of delivering the fluidic composition to a location below the dermal barrier from about 50 μm to about 4000 μm, from about 250 μm to about 2000 μm, or from about 350 μm to about 1000 μm in depth.
Embodiment 7. The device of any one of claims 1-6, wherein the device is capable of delivering the fluidic composition to a location below the dermal barrier and proximate to the lymphatic vasculature of the subject.
Embodiment 8. The device of any one of claims 1-7, wherein the dermal barrier comprises the stratum corneum of the subject.
Embodiment 9. The device of any one of claims 1-7, wherein the dermal barrier comprises a portion of the epidermis of the subject.
Embodiment 10. The device of any one of claims 1-7, wherein the dermal barrier comprises the entire thickness of epidermis of the subject.
Embodiment 11. The device of any one of claims 1-7, wherein the dermal barrier comprises at least a portion of the dermis of the subject.
Embodiment 12. The device of any one of claims 1-11, wherein the device is capable of delivering the fluidic composition having a viscosity from about 1 centipoise to about 100 centipoise.
Embodiment 13. The device of any one of claims 1-12, wherein the device is capable of delivering the fluidic composition having a viscosity from about 1 centipoise to about 5 centipoise.
Embodiment 14. The device of any one of claims 1-13, wherein the device is capable of delivering the fluidic composition having a bioactive (diagnostic or therapeutic) agent in a concentration of from about 5 mg/mL to about 100 mg/mL.
Embodiment 15. The device of any one of claims 1-14, wherein the plurality of protrusions comprises from about 4 to about 3,000 protrusions
Embodiment 16. The device of any one of claims 1-15, wherein the plurality of protrusions comprises from about 100 to about 2,500 protrusions
Embodiment 17. The device of any one of claims 1-16, wherein the plurality of protrusions comprises about 100 protrusions.
Embodiment 18. The device of any one of claims 1-17, wherein the plurality of protrusions comprises about 324 protrusions.
Embodiment 19. The device of any one of claims 1-18, wherein the device is capable of delivering the flow composition at the flow rate per protrusion ranging from about 0.1 μl/hour to about 10 μl/hour, about 0.5 μl/hour to about 7.5 μl/hour, about 1 μl/hour to about 5 μl/hour, 1.5 μl/hour to about 5 μl/hour, or about 0.15 μl/hour to about 1.5 μl/hour.
Embodiment 20. The device of any one of claims 1-19, wherein the device is capable of delivering the flow composition at the flow rate per protrusion is about 0.1 μl/hour, 0.15 μl/hour, 0.5 μl/hour, 1 μl/hour, 1.5 μl/hour, 2 μl/hour, 5 μl/hour, 7.5 μl/hour, or 10 μl/hour.
Embodiment 21. The device of any one of claims 1-20, wherein the device is capable of delivering the flow composition at the overall device flow rate ranging from about 1 μl/hour to about 25,000 μl/hour, from about 10 μl/hour to about 20,000 μl/hour, from about 100 μl/hour to about 25,000 μl/hour, from about 200 μl/hour to about 15,000 μl/hour, from about 500 μl/hour to about 10,000 μl/hour, or from about 1000 μl/hour to about 5,000 μl/hour.
Embodiment 22. The device of any one of claims 1-21, wherein the device is capable of delivering the flow composition at the overall device flow rate of about 10 μl/hour, 100 μl/hour, 200 μl/hour, 500 μl/hour, 1000 μl/hour, 1,500 μl/hour, 2,000 μl/hour, 2,500 μl/hour, 3,000 μl/hour, 5,000 μl/hour, 10,000 μl/hour, or 20,000 μl/hour.
Embodiment 23. The device of any one of claims 1-22, wherein the device is capable of delivering the flow composition at the overall device flow rate of 100 μl/hour.
Embodiment 24. The device of any one of claims 1-22, wherein the device is capable of delivering the flow composition at the overall device flow rate of 500 μl/hour.
Embodiment 25. The device of any one of claims 1-24, wherein the protrusions of the plurality are arranged in an approximately evenly spaced pattern.
Embodiment 26. The device of any one of claims 1-25, wherein the protrusions are arranged in 2-50 rows and 2-50 columns in an equidistant manner.
Embodiment 27. The device of any one of claims 1-26, wherein the protrusions are arranged in 10 rows and 10 columns, and the device is capable of delivering the flow composition with the overall device flow rate of about 100 μl/hour.
Embodiment 28. The device of any one of claims 1-26, wherein the protrusions are arranged in 18 rows and 18 columns, and the device is capable of delivering the flow composition with the overall device flow rate of about 500 μl/hour.
Embodiment 29. The device of any one of claims 1-28, wherein the flow rate does not change for at least a predetermined time period.
Embodiment 30. The device of any one of claims 1-29, wherein the flow rate of the fluidic composition increases or decreases for a predetermined time period.
Embodiment 31. The device of any one of claims 1-30, wherein the flow rate changes over time in a sinusoidal, parabolic, triangular, or step-wise manner.
Embodiment 32. The device of any one of claims 1-31, wherein each of the protrusions has the height ranging from 1 μm to 1 mm, about 200 to about 800 μm, between about 250 to about 750 μm, or between about 300 to about 600 μm.
Embodiment 33. The device of any one of claims 1-32, wherein the protrusions have a cross-sectional dimension perpendicular to the height, wherein an aspect ratio of the height to the cross-sectional dimension is greater than 2, 3 or 4.
Embodiment 34. The device of any one of claims 1-33, wherein the fluidic path in the protrusions has a length and a cross-sectional dimension perpendicular to the length, wherein an aspect ratio of the length to the cross-section dimension ranges from about 1 to about 50, about 5 to about 40, or about 10 to about 20 in average.
Embodiment 35. The device of any one of claims 1-34, wherein the cross-sectional dimension of the fluidic path ranges from about 1 μm to about 100 μm, about 5 μm to about 50 μm, or about 10 μm to about 30 μm.
Embodiment 36. The device of any one of claims 1-35, wherein the nanostructures comprise a height and a cross-sectional dimension, and at least a portion of the nanostructures have one or more of the following characteristics:
   a) center-to-center spacing of from about 50 nanometers to about 1 micrometer;
   b) a height of from about 10 nanometers to about 20 micrometers;
   c) an aspect ratio of the height to the cross-sectional dimension from about 0.15 to about 30;
   d) the plurality of nanostructures constitute a nanopattern having a fractal dimension of greater than about 1;
   e) the surface of the protrusion comprising a plurality of nanostructures having an average surface roughness ranging from about 10 nm to about 200 nm; and/or
   f) an effective compression modulus ranging from about 4 MPa to about 320 MPa.
Embodiment 37. The device of any one of claims 1-36, wherein the nanopatterned layer further comprises a plurality of additional nanostructures having a cross-sectional dimension less than the cross-sectional dimension of the nanostructures.
Embodiment 38. The device of any one of claims 1-37, wherein the nanopatterned layer comprises a polyether ether ketone (PEEK) film.
Embodiment 39. The device of any one of claims 1-38, comprising a cartridge assembly including a reservoir component having an upper cavity and an opposing lower cavity coupled together in flow communication with the fluidic block.
Embodiment 40. The device of any one of claims 1-39, comprising a reservoir for holding the fluidic composition located exterior of the device, and fluidically connected to the fluidic block.
Embodiment 41. The device of any one of claims 1-40, wherein the collet assembly comprises a collet lock coupled to a collet.
Embodiment 42. The device of any one of claims 1-41, wherein the collet lock is permanently coupled to the collet, optionally wherein the coupling is via a UV-curable adhesive.
Embodiment 43. The device of any one of claims 1-42, further comprising an attachment band assembly configured to couple to the collet assembly to facilitate contact with a surface of the subject's skin sufficient for penetration of the plurality of protrusions into the surface of the subject's skin and across the dermal barrier.
Embodiment 44. The device of claim 43, wherein the attachment band assembly comprises:
   a. an annular body comprising a wall defining a hollow inner space and a coupling member to engage with a corresponding coupling member of the collet, wherein the annular body is configured to attach to the collet of the collet assembly;
   b. a strap assembly removably engaged with the annular body and comprising a hoop-and-loop type fastening strap such that in use, the strap is threaded through a portion of the annular body and folded back to tighten the strap around the skin of the subject.
Embodiment 45. The device of any one of claims 1-44, wherein the controller assembly includes a plunger member positionable in a range from a first position proximal to the plenum, to a second position distant from the plenum; and a bias assembly positioned between the plenum and the plunger member, the bias assembly being configured to apply a pressure to the plunger member.
Embodiment 46. The device of claim 45, wherein the pressure applied to the plunger member by the biasing assembly is transmitted to the plenum and facilitates displacing the fluidic composition into the fluidic block.
Embodiment 47. The device of any one of claims 1-46, wherein the controller assembly includes an external infusion pump and a tubing system to provide a pressured flow of the fluidic composition from a reservoir for holding the fluidic composition located exterior of the device into the device and through the plenum to the fluidic block.
Embodiment 48. The device of claim 47, wherein the external infusion pump is a syringe pump, an elastomeric pump, or a peristaltic pump.
Embodiment 49. The device of claim 48, wherein the external infusion pump is portable.
Embodiment 50. The device of any one of claims 1-49, wherein the device is capable of delivering the flow composition with a protrusion to protrusion variability of a flow rate less than 50%, less than 40%, less than 30%, less than 20%, or less than 10% over at least 75% of the protrusions.
Embodiment 51. The device of any one of claims 1-50, wherein the device is capable of delivering the flow composition with a protrusion to protrusion variability of the flow rate being about 10% or less.
Embodiment 52. A method for delivering a fluidic composition across a dermal barrier of a subject comprising:
   inserting the plurality of protrusions of at least one device of any of the preceding claims across the dermal barrier of the subject; and
   transporting the fluidic composition through the fluidic path of the plurality of protrusions to a location below the dermal barrier.
Embodiment 53. The method of claim 52, wherein the fluidic composition is delivered at a flow rate greater than about 0.4 μl/hour, or at a flow rate ranging from about 0.4 μl/hour to about 25,000 μl/hour.
Embodiment 54. A method for delivering a fluidic composition across a dermal barrier of a subject comprising:
   penetrating the dermal barrier with a device having a plurality of protrusions with a nanopatterned layer comprising nanostructures overlaid thereon; and
   transporting the fluidic composition through the fluidic path of the plurality of protrusions to a location below the dermal barrier,
   wherein the number of protrusions in the plurality of protrusions is from about 4 to about 2,500 protrusions, and the fluidic composition is transported to a location below the dermal barrier at a flow rate greater than about 0.1 μl/hour per protrusion, or at a flow rate ranging from about 0.1 μl/hour to about 10 μl/hour per protrusion.
Embodiment 55. The method of any one of claims 52-54, further comprising transporting the fluidic composition to lymphatic vasculature of the subject.
Embodiment 56. The method of any one of claims 52-55, comprising increasing permeability of the lymphatic vasculature wherein the nanostructures are in contact with, or proximate to, epithelial cells of the subject, thereby opening intercellular junctions between the epithelial cells and facilitating the flow of the fluidic composition during transport to the location below the dermal barrier.
Embodiment 57. A method for delivering a fluidic composition across a dermal barrier of a subject comprising:
   applying more than one device of any of the preceding claims at two or more positions of a subject; and
   transporting the fluidic composition through the fluidic path of the plurality of protrusions to a location below the dermal barrier.
Embodiment 58. A method for delivering a fluidic composition across a dermal barrier of a subject comprising:
   placing a first device of any of the preceding claims on the skin of a subject at a first position proximate to a first location below the dermal barrier; and
   placing a second device of any of the preceding claims on the skin of the subject at a second position proximate to a second location below the dermal barrier; and
   in one or more steps, inserting the plurality of protrusions of the first device into the subject to a depth whereby an end of at least one of the protrusions is proximate to the first location, and inserting the plurality of protrusions of the second device into the subject to a depth whereby an end of at least one of the protrusions is proximate to the second location; and
   in one or more steps, administering via the protrusions of the first device a first dose of the fluidic composition into the first location; and administering via the protrusions of the second device a second dose of the fluidic composition into the second location.
Embodiment 59. The method of claim 58, wherein administering the first dose and administering the second dose is simultaneous.
Embodiment 60. The method of claim 58, wherein administering the first dose and administering the second dose partially overlap in time.
Embodiment 61. The method of claim 58, wherein administering the first dose and administering the second dose is sequential.
Embodiment 62. The method of any one of claims 58-61, wherein the first and second devices are different devices.
Embodiment 63. The method of any one of claims 58-61, wherein the first and second devices are the same device.
Embodiment 64. The method of any one of claims 58-63, wherein administering the doses cumulatively provides a therapeutically effective dose.
Embodiment 65. The method of any one of claims 58-64, wherein the first location and the second location are on different limbs of the subject.
Embodiment 66. The method of any one of claims 58-65, wherein the first location and the second location are each independently proximate to the hands or the feet of the patient.
Embodiment 67. The method of any one of claims 58-66, wherein the administration step is conducted for at least 4, 6, 8, 10, 12, 16, 24, 36, 48 or 72 hours.
Embodiment 68. The method of any one of claims 58-67, wherein the device is placed at a location on the skin of the subject having lymphatic capillaries and/or vessels that deliver lymph directly into the lymphatic system in an inflammatory locus in the patient comprising lymph nodes, lymph capillaries, lymph vessel, lymph organs or any combination thereof.

### BRIEF DESCRIPTION OF THE DRAWINGS

FIG. 1 is an exploded, sectional view of an embodiment of a fluid delivery apparatus.

FIGS. 2A-2B are sectional views of the exemplary fluid delivery apparatus shown in FIG. 1 in a non-activated configuration and an activated configuration, respectively.

FIGS. 3A-3B are perspective views of the exemplary fluid delivery apparatus shown in FIG. 1 in a non-activated configuration and an activated configuration, respectively.

FIG. 4 is an exploded, perspective view of the collet assembly comprising a collet and a collet lock, of the fluid delivery apparatus shown in FIG. 1.

FIG. 5 is a sectional view of a plenum assembly of the fluid delivery apparatus shown in FIG. 1 with the fluid distribution assembly connected thereto.

FIG. 6 is an exploded, perspective view of the plenum assembly with the fluid distribution assembly connected thereto.

FIGS. 7A-7B are top and bottom views of a sleeve component of the plenum assembly.

FIG. 8 is a section view of the sleeve component taken about line A-A shown in FIG. 7A.

FIG. 9 is an enlarged view of section B shown in FIG. 9.

FIG. 10 is a side view of the sleeve component from direction C shown in FIG. 7A.

FIGS. 11A-11D show an embodiment of a plenum component of the plenum assembly. FIG. 11A is a top view of a plenum component of the plenum assembly; FIG. 11B is a section view of the plenum component taken about line A-A shown in FIG. 11A; FIG. 11C is an exploded view of section B shown in FIG. 11B; FIG. 11D is an exploded view of section C shown in FIG. 11B.

FIGS. 12A-12D show another embodiment of a plenum component of a fluid delivery apparatus according to an embodiment of the present disclosure, which includes tubing system connected to an external pump. FIG. 12A is a top view of a plenum component of the plenum assembly; FIG. 12B is a section view of the plenum component taken about line A-A shown in FIG. 12A; FIG. 12C is an exploded view of section B shown in FIG. 12B; FIG. 12D is an exploded view of section C shown in FIG. 12B.

FIGS. 13A-13B show a bottom view of an embodiment of the plenum component. FIG. 13B is an exploded view of Section D of FIG. 13A.

FIG. 14 is an exploded, schematic of a plenum cap assembly of the fluid delivery apparatus;

FIG. 15 is a top view of the plenum cap assembly, showing a first adhesive layer;

FIG. 16 is a top view of a second adhesive layer of the plenum cap assembly;

FIG. 17 is a top view of a third adhesive layer of the plenum cap assembly;

FIG. 18A is an exploded perspective view of a plurality of protrusions, a PSA layer, and a nanopatterned layer of the fluid distribution assembly of an embodiment of a fluidic distribution assembly.

FIG. 18B is a top view of the fluid distribution assembly of FIG. 18A having a 18×18 array of protrusions.

FIG. 19A for a schematic cross-sectional view of a fluidic distribution assembly.

FIG. 19B show a top view of the distribution manifold of a fluidic distribution assembly.

FIG. 20 is a sectional view of a cartridge assembly of the fluid delivery apparatus according to an embodiment of the disclosure;

FIG. 21 is an exploded schematic of the cartridge assembly of FIG. 20;

FIG. 22 is a sectional view of a cap assembly of the fluid delivery apparatus according to an embodiment of the disclosure;

FIG. 23 is an exploded, perspective view of a mechanical controller assembly according to an embodiment of the disclosure.

FIG. 24 is a perspective sectional view of the assembled mechanical controller assembly of FIG. 23.

FIGS. 25A-25E show the housing component of the mechanical controller assembly shown in FIG. 23. FIG. 25A shows a top view of the housing component, and FIG. 25B show a sectional view of the housing component taken about line A-A of FIG. 25A. FIG. 25C shows a bottom view of the housing component. FIG. 25D shows a sectional view of the housing component taken about line B-B of FIG. 25 C. FIG. 25E shows an exploded view of section C shown in FIG. 25D.

FIGS. 26A-26E show the insert component of the mechanical controller assembly shown in FIG. 23. FIGS. 26A-26B show top and bottom views respectively of the insert component. FIG. FIG. 26C shows a sectional view of the insert component taken about line A-A shown in FIG. 26B. FIG. 26D shows a sectional view of the insert component taken about line C-C of FIG. 26C. FIG. 26E shows a sectional view of the insert component taken about line B-B of FIG. 26B.

FIGS. 27A-27C show the plunger component of the mechanical controller assembly shown in FIG. 23. FIG. 27A shows a top view of the plunger component. FIG. 27B shows a side view of the plunger component; FIG. 27C shows a sectional view of the plunger component taken about line A-A shown in FIG. 27B.

FIGS. 28A-28B show an attachment band assembly according to an embodiment of the disclosure. FIG. 28A shows a top view of the attachment band assembly. FIG. 28B shows a sectional view of the attachment band assembly taken bout line A-A of FIG. 28A.

FIGS. 29A-29B shows an attachment ring of the attachment band assembly shown in FIGS. 28A-28B. FIG. 29A is a top view of the attachment ring with the wings 431. FIG. 29B is a sectional view of the attachment ring taken about line A-A of FIG. 29A.

FIG. 30A-30C show an applicator of the fluid delivery apparatus according to an embodiment of the present disclosure. FIG. 30A shows a perspective view of an applicator of the fluid delivery apparatus. FIG. 30B shows a front sectional view of the applicator shown in FIG. 30A. FIG. 30C shows a side sectional view of the applicator shown in FIG. 30A.

FIG. 31 shows an overview of a plurality of subassembly components of a fluid delivery apparatus according to an embodiment of the present disclosure.

Unless otherwise indicated, the drawings provided herein are meant to illustrate features of embodiments of the disclosure or results of representative experiments illustrating some aspects of the subject matter disclosed herein. These features and/or results are believed to be applicable in a wide variety of systems comprising one or more embodiments of the disclosure. As such, the drawings are not meant to include all additional features known by those of ordinary skill in the art to be required for the practice of the embodiments, nor are they intended to be limiting as to possible uses of the methods disclosed herein.

### DETAILED DESCRIPTION

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents, which may be included within the invention as defined by the appended claims. The section headings used herein are for organizational purposes only and are not to be construed as limiting the desired subject matter in any way. In the event that any literature incorporated by reference contradicts any term defined in this specification, this specification controls.

In the following specification and the claims, reference will be made to a number of terms, which shall be defined to have the following meanings. The singular forms “a,” “an,” and “the” include plural references unless the context clearly dictates otherwise. The terms “comprising,” “including,” and “having” are intended to be inclusive and mean that there may be additional elements other than the listed elements. “Optional” or “optionally” means that the subsequently described event or circumstance may or may not occur, and that the description includes instances where the event occurs and instances where it does not.

“Or” is used in the inclusive sense, i.e., equivalent to “and/or,” unless the context requires otherwise.

Approximating language, as used herein throughout the specification and claims, may be applied to modify any quantitative representation that could permissibly vary without resulting in a change in the basic function to which it is related. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. “About” indicates a degree of variation that does not substantially affect the properties of the described subject matter, e.g., within 10%, 5%, 2%, or 1%. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed considering the number of reported significant digits and by applying ordinary rounding techniques. “Approximately” and “substantially” are synonymous with “about.”

As used herein, positional terms such as upward, downward, upper, lower, top, bottom, and the like are used only for convenience to indicate relative positional relationships.

### I. Definitions

As used herein, a “dermal barrier” means a portion of a subject's skin structure. The dermal barrier may include one or more layers of the skin (such as the stratum corneum, epidermis, and/or dermis). In some embodiments, the dermal barrier comprises the stratum corneum of the subject. In some embodiments, the dermal barrier comprises a portion of the epidermis of the subject. In some embodiments, the dermal barrier comprises the entire thickness of epidermis of the subject. In some embodiments, the dermal barrier comprises at least a portion of the dermis of the subject.

As used herein, “lymphatic vasculature” includes any vessel or capillary that carries fluid toward a lymph node or from a lymph node toward a blood vessel. “Proximate to the lymphatic vasculature” means sufficiently close to the lymphatic vasculature for material from a fluidic composition to be taken up into the lymphatic vasculature.

As used herein, an “aspect ratio” means the ratio of the height or length of a structure to the cross-sectional dimension perpendicular to the height or length (e.g., width or diameter) of the structure. In instances in which the cross-sectional dimension (e.g., diameter of the protrusion having a conical shape) varies over the height, the aspect ratio is determined based on the average cross-sectional dimension unless otherwise indicated. When the term “height” is used to describe a fluidic path defined in a protrusion, the height may encompass a length of a fluidic path regardless of whether the fluidic path is defined in the center or off-center in the protrusion. In other words, in some instances, the height of the protrusion may not be necessarily the same as the height (or length) of the fluidic path defined therein.

The terms “medicament”, “medication”, “medicine”, “therapeutic agent” and “drug” are used interchangeably herein and describe a pharmaceutical composition or product intended for the treatment of a medical condition having at least one symptom. The pharmaceutical composition or product will have a physiological effect on the patient when it is introduced into the body of a patient. The pharmaceutical composition can be in any suitable formulation unless a specific formulation type is required or disclosed. In some instances, the medicament will be approved by the US FDA while in other instances it may be experimental (e.g., in clinical or pre-clinical trials) or approved for use in a country other than the United States (e.g., approved for use in China or Europe). In instances where these terms are used, it is understood that they refer to both singular and plural instances. In some embodiments herein, two or more medicaments may be used in a form of combination therapy. In all cases, the selection of the proper medicament (singular or plural) will be based on the medical condition of the patient and the assessment of the medical professional administering, supervising and/or directing the treatment of the patient. Combination therapies are sometimes more effective than a single agent and used for many different medical conditions. It is understood that combination therapies are encompassed herein and envisioned with the subject matter disclosed.

An “effective amount” or a “therapeutically effective dose” in reference to a medicament is an amount sufficient to treat, ameliorate, or reduce the intensity of at least one symptom associated with the medical condition. In some aspects of this disclosure, an effective amount of a medicament is an amount sufficient to effect a beneficial or desired clinical result including alleviation or reduction in one or more symptoms of a medical condition. In some embodiments, an effective amount of the medicament is an amount sufficient to alleviate all symptoms of a medical condition. In some aspects, a dose of the therapeutic agent will be administered that is not therapeutically effective by itself. In these aspects, multiple doses may be administered to the patient either sequentially (using the same device or different devices) or simultaneously such that the combination of the individual doses is therapeutically effective. For simultaneous administration, additional medical devices comprising a plurality of protrusions or an entirely different route of administration may be used.

The term “patient” as used herein refers to a warm blooded animal such as a mammal which is the subject of a medical treatment for a medical condition that causes at least one symptom. It is understood that at least humans, dogs, cats, and horses are within the scope of the meaning of the term. Preferably, the patient is human.

As used herein, the terms “distal” and “proximal” are used in their anatomical sense. Distal means a given position or structure is situated farther from the center of the body or point of attachment of the limb when compared to another position or structure. Proximal is the opposite of distal. Proximal means a given position or structure is situated closer to the center of the body or point of attachment of the limb when compared to another position or structure. For example, the wrist is distal to the elbow and the shoulder is proximal to the elbow.

As used herein, the term “treat” or “treatment”, or a derivative thereof, contemplates partial or complete amelioration of at least one symptom associated with the medical condition of the patient, including but not limited to slowing or arresting the worsening of a symptom that would occur in the absence of treatment. “Preventing” a symptom or medical condition from occurring is considered a form of treatment. “Reducing” the incidence of a symptom or medical condition is considered a form of treatment.

As used herein, “bioavailability” means the total amount of a given dosage of the administered agent that reaches the blood compartment measured as a ratio of (AUC/dose) for a given route of administration/(AUC/dose) for intravenous administration with the area under the curve (AUC) in a plot of concentration vs. time.

Cₘₐₓ refers to the maximum concentration that a medicament achieves in the plasma or tissue of a patient after the medicament has been administered while Cₜ refers to the concentration that a medicament achieves at a specific time (t) following administration. Unless otherwise stated, all discussion herein is in regard to pharmacokinetic parameters in plasma.

The AUCₜ refers to the area under the plasma concentration time curve from time zero to time t following administration of the medicament.

The AUC_{∞} refers to the area under the plasma concentration time curve from time zero to infinity (infinity meaning that the plasma concentration of the medicament is below detectable levels).

Tₘₐₓ is the time required for the concentration of a medicament to reach its maximum blood plasma concentration in a patient following administration. Some forms of administration of a medicament will reach their Tₘₐₓ slowly (e.g., tablets and capsules taken orally) while other forms of administration will reach their Tₘₐₓ almost immediately (e.g., subcutaneous and intravenous administration).

“Steady state” refers to the situation where the overall intake of a drug is approximately in dynamic equilibrium with its elimination.

A discussion of various pharmacokinetic parameters and the methods of measuring and calculating them can be found in Clinical Pharmacokinetics and Pharmacodynamics: Concepts and Applications, M. Rowland and T. N. Tozer, (Lippincott, Williams & Wilkins, 2010) which is incorporated by reference for its teachings thereof.

### II. Fluid Delivery Device

In some embodiments, a device for delivering a fluidic composition across a dermal barrier of a subject is provided. In some embodiments, the device may comprise a fluid distribution assembly. The fluid distribution assembly may comprise a base, a plurality of protrusions defined on the base, wherein each of the protrusions has a tip and a height at a microscale with a fluidic path defined therein along the height from the base, a nanopatterned layer comprising a plurality of nanostructures and covering a surface of the plurality of protrusions. The fluid distribution assembly may further comprise a gasket comprising a pressure-sensitive adhesive (PSA) layer. The fluid distribution assembly may further comprise a fluidic block configured to be fluidically connected with the fluidic path of the protrusions and to controllably distribute the fluidic composition among the plurality of protrusions through the fluidic path.

In some embodiments, the device may further comprise a plenum assembly slidably coupled to the fluidic block and configured to hold the fluidic distribution assembly. In some embodiments, the device may further comprise a collet assembly constituting the housing of the device and configured to contact a surface of the subject's skin sufficient for penetration of the plurality of protrusions into the surface of the subject's skin and across the dermal barrier. In some embodiments, the device may further comprise a controller assembly slidably coupled to the fluidic block and configured to control the flow of the fluidic composition during delivery of the fluidic composition through the plurality of protrusions.

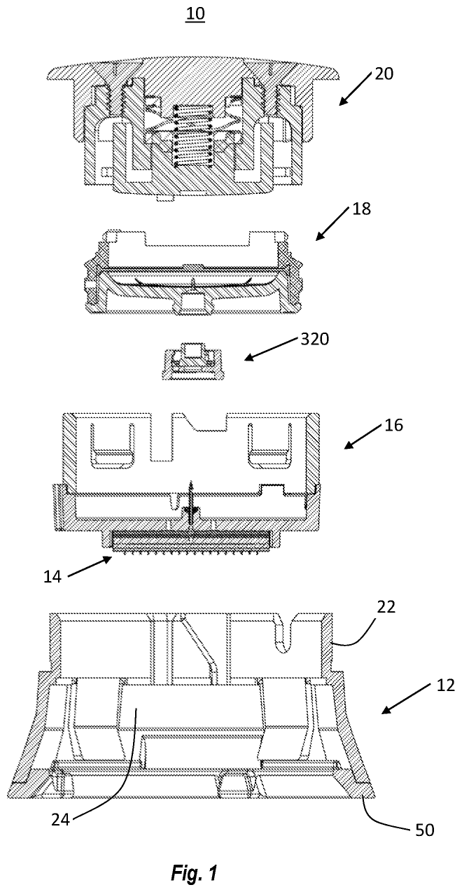

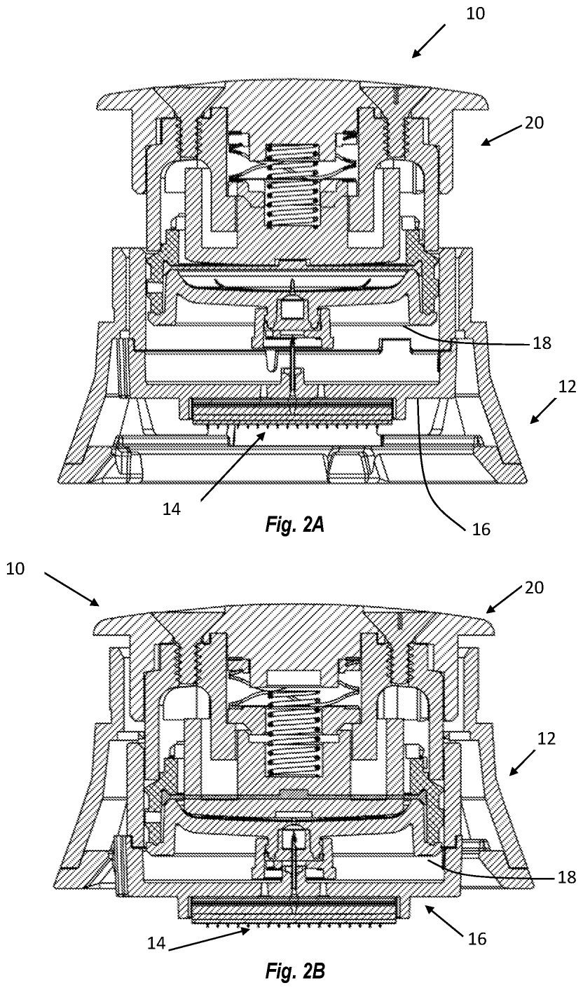

Certain embodiments of the fluid delivery apparatus are illustrated in the drawings. FIG. 1 is an exploded, sectional view of fluid delivery apparatus 10. FIG. 2A is a sectional view of a fluid delivery apparatus 10 in a non-activated configuration. FIG. 2B is a sectional view of the fluid delivery apparatus 10 in an active configuration. In one embodiment, the fluid delivery apparatus 10 includes a plurality of subassembly components coupled together to form the fluid delivery apparatus 10, including a collet assembly 12 and a fluid distribution assembly 14. The collet assembly 12 and the fluid distribution assembly 14 are indicated generally by their respective reference numbers. As shown in FIG. 1, the fluid distribution assembly 14 includes a plurality of additional subassembly components, including a plenum assembly 16, a cartridge assembly 18, a cap assembly 320, and a mechanical controller assembly 20. Each of the collet assembly 12, the fluid distribution assembly 14, the plenum assembly 16, the cartridge assembly 18, the cap assembly 320, and the mechanical controller assembly 20 is indicated generally in the accompanying drawings by their reference numbers. The collet assembly 12 forms the body or housing of the fluid delivery apparatus 10 and is slidably coupled to the fluid distribution assembly 14. To form the fluid distribution assembly 14, the cap assembly 320 is coupled to the cartridge assembly 18, and the cartridge assembly 18 is slidably coupled to the plenum assembly 16. In addition, the mechanical controller assembly 20, as explained in more detail below, is coupled to the cartridge assembly 18.

In some embodiments, a device for delivering a fluidic composition across a dermal barrier of a subject is provided. In some embodiments, the device may comprise a fluid distribution assembly as described herein, a plenum assembly as described herein, slidably coupled to the fluidic block and configured to hold the fluidic distribution assembly, a cartridge assembly comprising a reservoir component includes an upper cavity and an opposing lower cavity coupled together in flow communication with the fluidic block via the cannula of the plenum assembly, a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and a controller assembly slidably coupled to the fluidic block and configured to control the flow of the fluidic composition during delivery of the fluidic composition through the plurality of protrusions. The controller assembly comprises a plunger member positionable in a range from a first position proximal to the plenum, to a second position distant from the plenum; and a bias assembly positioned between the plenum and the plunger member, the bias assembly being configured to apply a pressure to the plunger member, wherein the pressure applied to the plunger member by the biasing assembly is transmitted to the plenum and facilitates displacing the fluidic composition into the fluidic block.

In some embodiments, a device for delivering a fluidic composition across a dermal barrier of a subject is provided. In some embodiments, the device may comprise a fluid distribution assembly as described herein, a plenum assembly as described herein, slidably coupled to the fluidic block and configured to hold the fluidic distribution assembly, a cartridge assembly comprising a reservoir component includes an upper cavity and an opposing lower cavity coupled together in flow communication with the fluidic block via the cannula of the plenum assembly, a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and an external infusion pump configured to control the flow of the fluidic composition during delivery of the fluidic composition through the plurality of protrusions.

In some embodiments, the device described herein is capable of delivering the fluidic composition to a location below the dermal barrier from about 50 μm to about 4000 μm, from about 250 μm to about 2000 μm, or from about 350 μm to about 1000 μm in depth. In some embodiments, the device described herein is capable of delivering the fluidic composition to a location below the dermal barrier and proximate to the lymphatic vasculature of the subject.

### A. Fluid Distribution Assembly

In some embodiments, a fluid distribution assembly may comprise a base, a plurality of protrusions defined on the base, and a nanopatterned layer covering a surface of the plurality of protrusions. Each of the protrusions has a tip and a height at a microscale with a fluidic path defined therein along the height from the base. The nanopatterned layer comprises a plurality of nanostructures, which will be described further herein. In some embodiments, the fluid distribution assembly also comprises a gasket comprising a pressure-sensitive adhesive (PSA) layer. In some embodiments, the fluid distribution assembly also comprise a fluidic distribution manifold. The fluidic distribution manifold is configured to be fluidically connected with the fluidic path of the protrusions and to controllably distribute the fluidic composition among the plurality of protrusions through the fluidic path. In some embodiments, a fluid distribution assembly may comprise a base, a plurality of protrusions defined on the base, a nanopatterned layer covering a surface of the plurality of protrusions, a gasket comprising a pressure-sensitive adhesive (PSA) layer, and fluidic distribution manifold.

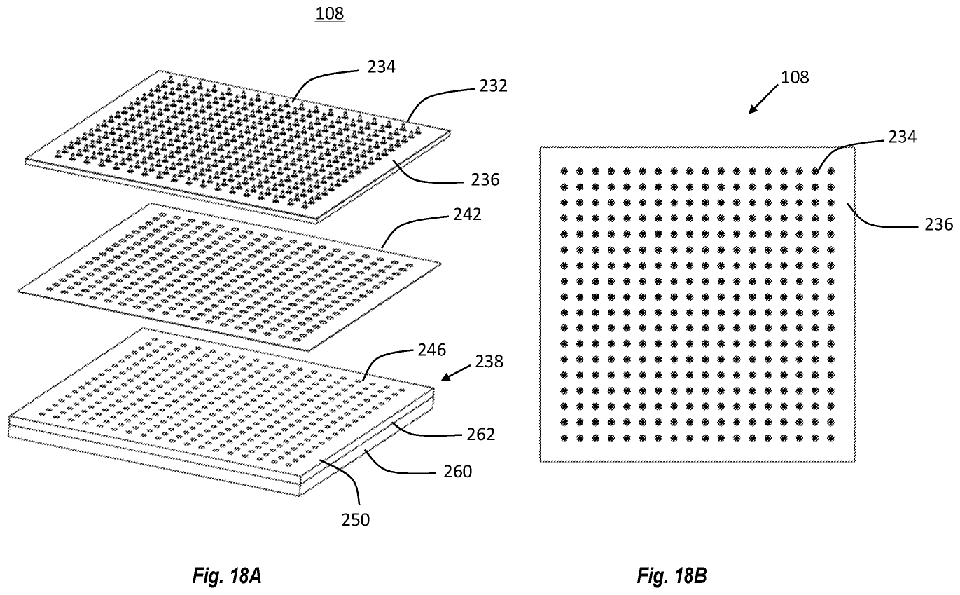

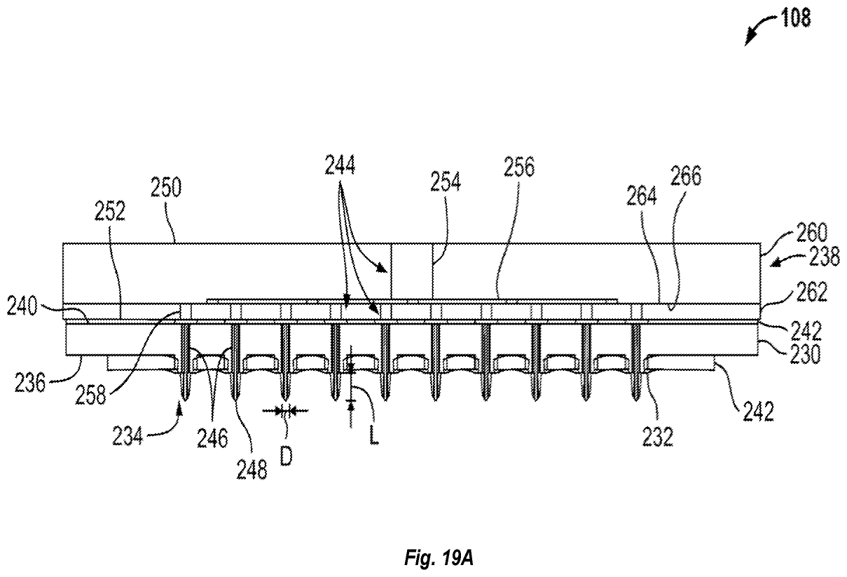

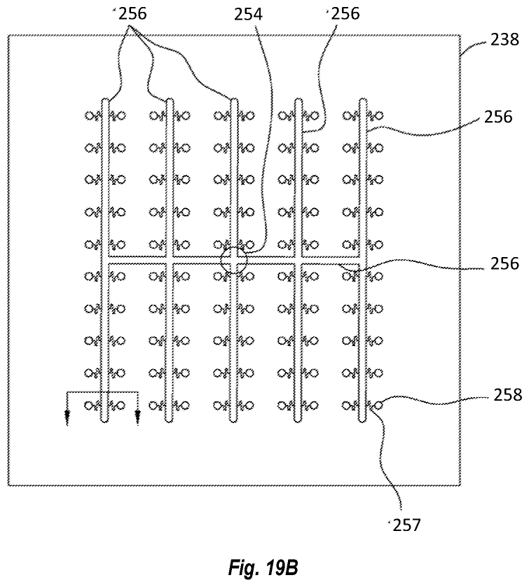

FIG. 18A is an exploded, schematic of an embodiment of the fluid distribution assembly 108 (14) of the fluid delivery apparatus 10 shown in FIG. 1. FIG. 18B is a top view of the fluid distribution assembly of FIG. 18A having a 18×18 array of protrusions.

FIG. 19A is a schematic cross-sectional view of the fluid distribution assembly according to an embodiment of the present disclosure. The fluid distribution assembly 108 is bonded to the plenum cap assembly by use of an adhesive layer. The fluid distribution assembly 108 includes a plurality of protrusions 234 and a nanopatterned layer 232 draped at least partially across a plurality of protrusions and a base of the fluid distribution assembly. Each of the protrusions 234 has a tip 248.

A fluidic distribution assembly may generally include any suitable number of protrusions. In some embodiments, the plurality of protrusions comprises at least about 4 protrusions. In some embodiments, the plurality of protrusions comprises from about 4 protrusions to 3,000 protrusions. In some embodiments, the plurality of protrusions comprises from about 4 to about 2,500 protrusions. In some embodiments, the plurality of protrusions comprises from about 100 to about 2,500 protrusions. In some embodiments, the plurality of protrusions comprises from about 25 to about 500 protrusions. In some embodiments, the plurality of protrusions comprises from about 60 to about 400 protrusions. In some embodiments, the plurality of protrusions comprises from about 80 to about 400 protrusions. In some embodiments, the plurality of protrusions comprises from about 100 to about 400 protrusions. In some embodiments, the number of protrusions in the plurality of protrusions is in a range from about 80 to about 400. In some embodiments, the fluidic distribution assay comprises 64 protrusions. In some embodiments, the fluidic distribution assay comprises 100 protrusions. In some embodiments, the fluidic distribution assay comprises 324 protrusions. In some embodiments, the fluidic distribution assay comprises 400 protrusions. In some embodiments, the fluidic distribution assay comprises 2,500 protrusions.

In some embodiments, the quantity of protrusions per unit area is in the range from about 10 protrusions per square centimeter (cm²) to about 1,500 protrusions per cm², such as from about 50 protrusions per cm² to about 1250 protrusions per cm², or from about 100 protrusions per cm² to about 500 protrusions per cm², or any other subranges therebetween.

The protrusions described herein need not be identical to one another. A plurality of protrusions may have various lengths, outer diameters, inner diameters, cross-sectional shapes, nanotopography surfaces, and/or spacing. For example, the protrusions may be spaced apart in a uniform manner, such as, for example, in a rectangular or square grid or in concentric circles. The spacing may depend on numerous factors, including height and width of the delivery structures, as well as the amount and type of an agent that is intended to be delivered through the delivery structures. In some embodiments, the spacing between each protrusions may be from about 1 μm to about 1500 μm, including each integer within the specified range. In some aspects, the spacing between each deliver structure may be about 200 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm or about 1500 μm. About as used in this context, “about” means ±50 μm.

In some embodiments, the protrusions of the plurality are arranged in an approximately evenly spaced pattern. In some embodiments, the protrusions are arranged in 2-50 rows and 2-50 columns in an equidistant manner. In some embodiments, the protrusions are arranged in 10 rows and 10 columns in an equidistant manner. In some embodiments, the protrusions are arranged in 18 rows and 18 columns in an equidistant manner.

In some embodiments, a plurality of protrusions extend outwardly from the base of the fluid distribution assembly. A fluidic path is defined in each protrusion along the height extending from the base. Each protrusion may be in a form of a conical or pyramidal shape, a rectangular or geometrically irregular shape, or a cylindrical, rectangular or geometrically irregular shape transitioning to a conical or pyramidal shape, or any other piercing or needle-like shape. The tip of each protrusion is disposed furthest away from the base of the fluid distribution assembly and defines the smallest dimension (e.g., diameter or cross-sectional width) of each protrusion.

Each protrusion may generally define any suitable height “H” between the base of the fluidic distribution assembly to its tip that is sufficient to allow the protrusions to penetrate the user's skin, i.e., penetrate the stratum corneum and pass into the epidermis of a user. It may be desirable to limit the height H of the protrusions such that the protrusions do not penetrate through the inner surface of the epidermis and into the dermis, which may advantageously facilitate minimizing pain for the user.

The overall height of the protrusions may vary depending on the location at which the fluid delivery apparatus is being used on the user. For example, and without limitation, the overall height of the protrusions for a fluid delivery apparatus to be used on a user's leg may differ substantially from the overall height of the protrusions for a fluid delivery apparatus to be used on a user's arm.

In some embodiment, each protrusion has a height H of less than about 1000 micrometers (μm), such as less than about 800 μm, or less than about 750 μm, or less than about 500 μm (e.g., an overall height ranging from about 200 μm to about 400 μm), or any other subranges therebetween. In some embodiments, each protrusion has the height ranging from 1 μm to 1 mm, about 200 to about 800 μm, from about 250 to about 750 μm, or from about 300 to about 600 μm. In some aspects, the length of each of the delivery structures may be from about 10 μm to about 1,000 μm. In some embodiments, each protrusion has the height from about 10 μm to about 5,000 μm, from about 50 to about 3,000 μm, from about 100 to about 1,500 μm, from about 150 to about 1,000 μm, from about 200 to about 800 μm, from about 250 to about 750 μm, or from about 300 to about 600 μm. The dimensions (height, cross-sectional dimension or the like) as described herein may be determined using standard geometric calculations known in the art.

Each protrusion may generally have any suitable aspect ratio (i.e., the height H over a cross-sectional width dimension D of each protrusion). The aspect ratio may be greater than 2, such as greater than 3 or greater than 4. In instances in which the cross-sectional width dimension (e.g., diameter) varies over the length of each protrusion, the aspect ratio may be determined based on the average cross-sectional width dimension. In some embodiments, an aspect ratio of the height to the cross-sectional dimension is greater than 2. In some embodiments, an aspect ratio of the height to the cross-sectional dimension is greater than 3. In some embodiments, an aspect ratio of the height to the cross-sectional dimension is greater than 4.

The fluidic path in each protrusion may be defined through the interior of the protrusion such that each protrusion forms a hollow shaft, or may extend along an outer surface of the protrusions to form a downstream pathway that enables the fluid to flow from the base of the fluid distribution assembly and through the fluidic paths, at which point the fluid may be delivered onto, into, and/or through the user's skin. The fluidic path may be configured to define any suitable cross-sectional shape, for example, without limitation, a semi-circular or circular shape. Alternatively, each fluidic path may define a non-circular shape, such as a V shape or any other suitable cross-sectional shape that enables the protrusions to function as described herein.

In some embodiments, the fluidic path in the protrusions has a length and a cross-sectional dimension perpendicular to the length. In some embodiments, the cross-sectional dimension of the fluidic path ranges from about 1 μm to about 100 μm, about 5 μm to about 50 μm, or about 10 μm to about 30 μm. In some embodiments, an aspect ratio of the length to the cross-section dimension ranges from about 1 to about 50, about 5 to about 40, or about 10 to about 20 in average

In some embodiments, the fluid distribution assembly comprises a nanopatterned layer comprising a plurality of nanostructures and covering a surface of the plurality of protrusions. In some embodiments, the nanostructures comprise a height and a cross-sectional dimension. In some embodiments, at least a portion of the nanostructures have center-to-center spacing of from about 50 nanometers to about 1 micrometer. In some embodiments, at least a portion of the nanostructures have a height of from about 10 nanometers to about 20 micrometers. In some embodiments, at least a portion of the nanostructures have an aspect ratio of the height to the cross-sectional dimension from about 0.15 to about 30. In some embodiments, the nanostructures constitute a nanopattern having a fractal dimension of greater than about 1. In some embodiments, at least a portion of the nanostructures have a surface comprising a plurality of nanostructures having an average surface roughness ranging from about 10 nm to about 200 nm. In some embodiments, at least a portion of the nanostructures have an effective compression modulus ranging from about 4 MPa to about 320 MPa. In some embodiments, the fluid distribution assembly comprises a nanopatterned layer comprising a plurality of nanostructures having one or more of the above described characteristics.

In some embodiments, the nanopatterned layer further comprises a plurality of additional nanostructures having a cross-sectional dimension less than the cross-sectional dimension of the nanostructures.

In some embodiments, the nanopatterned layer may be fabricated from a polymeric film, or the like, and coupled to the fluid distribution assembly using an additional adhesive layer. In other embodiments, the draped membrane may include an embossed or nano-imprinted, polymeric (e.g., plastic) film, or a polyether ether ketone (PEEK) film, or any other suitable material, such as a polypropylene film.

In some embodiments, the fluid distribution assembly may be fabricated from a rigid, semi-rigid, or flexible sheet of material, for example, without limitation, a metal material, a ceramic material, a polymer (e.g., plastic) material, or any other suitable material that enables the array of protrusions 230 to function as described herein. For example, in one embodiment, the fluid distribution assembly may be formed from silicon by way of reactive-ion etching, or in any other suitable fabrication technique.

In some embodiments, a gasket comprising a pressure-sensitive adhesive (PSA) layer is provided between the nanopatterned layer and the surface of the plurality of protrusions, providing support. The PSA layer is formed from an adhesive material (e.g., ARcare® 93445).

In some embodiments, the fluid distribution assembly includes a fluidic distribution manifold that extends across a surface of a base of the fluid distribution assembly. The fluidic distribution manifold may be bonded thereto by an adhesive layer. The fluid distribution manifold may include a fluid distribution network for supplying a fluidic composition to the fluidic path in one or more protrusions, for example, as depicted in FIG. 19B. The fluid distribution network is configured to provide uniform supply of a fluidic composition to the fluidic path in each composition.

In some embodiments, the fluidic distribution network includes a plurality of channels and/or apertures extending between a top surface and a bottom surface of the distribution manifold. The channels and/or apertures include a centrally-located inlet channel coupled in flow communication with a plurality of supply channels and the plenum cap assembly. In some embodiment, the supply channels facilitate distributing a fluid supplied by the inlet channel across an area of the distribution manifold. Each of the supply channels is coupled in flow communication to a plurality of resistance channels. The resistance channels extend away from the supply channels and are formed to facilitate an increase in the resistance of the fluid distribution network to the flow of the fluid. Each resistance channel may be coupled in flow communication to an outlet channel. Each outlet channel is aligned with a respective protrusion for distributing the fluid through the fluidic path. In some embodiments, the resistance channels may be formed in any configuration that enables the distribution manifold to function as described herein.

As depicted in FIG. 19A, in some embodiment, the distribution manifold is formed by bonding a base substrate 260 including the inlet channel 254 formed through the base substrate 260, and the supply channels 256 and the resistance channels (not shown) formed in a bottom surface 264, to a cover substrate 262 including the outlet channels 258 formed therethrough.

In some embodiments, the base substrate and the cover substrate of the distribution fold may comprise a glass material. In some embodiments, the base substrate and the cover substrate of the distribution fold may comprise silicon. The base substrate and the cover substrate may be fabricated from different materials of any combination that enables the distribution manifold to function as described herein. In one embodiment, the base substrate may comprise glass and the cover substrate may comprise silicon.

The inlet channel may be formed in the substrate by drilling, cutting, etching, and or any other manufacturing technique for forming a channel or aperture through substrate. In some embodiment, the supply channels and the resistance channels are formed in the bottom surface of the substrate using an etching technique. For example, in one embodiment, wet etching, or hydrofluoric acid etching, is used to form the supply channels and the resistance channels. In another suitable embodiment, Deep Reactive Ion Etching (DRIE or plasma etching) may be used to create deep, high density, and high aspect ratio structures in substrate. Alternatively, the supply channels and resistance channels can be formed in bottom surface using any fabrication process that enables the distribution manifold to function as described herein. In the exemplary embodiment, the outlet channels are formed through the cover substrate by drilling, cutting, etching, and or any other manufacturing technique for forming a channel or aperture through substrate.

In some embodiment, the base substrate and the cover substrate are bonded together in face-to-face contact to seal the edges of the supply channels and the resistance channels of the distribution manifold. In one embodiment, direct bonding, or direct aligned bonding, is used by creating a prebond between the two substrates. The prebond can include applying a bonding agent to the bottom surface of the substrate and a top surface of the cover substrate before bringing the two substrates into direct contact. The two substrates are aligned and brought into face-to-face contact and annealed at an elevated temperature. In another suitable embodiment, anodic bonding is used to form the distribution manifold. For example, an electrical field is applied across the bond interface at surfaces, while the substrates are heated. In an alternative embodiment, the two substrates may be bonded together by using a laser-assisted bonding process, including applying localized heating to the substrates to bond them together.

FIG. 19B is a schematic plan view of the distribution manifold 238 for use with the fluidic distribution assembly according to an embodiment of the present disclosure. The distribution manifold 238 includes the fluid distribution network 244 that includes, for example, a plurality of channels and/or apertures extending between a top surface 250 and a bottom surface 252 of the distribution manifold 238. The channels and/or apertures include a centrally-located inlet channel 254 coupled in flow communication with a plurality of supply channels 256, and the fluid passage 86 (shown in FIG. 1). In the exemplary embodiment, the plurality of supply channels 256 include 5 substantially parallel, equispaced channels extending longitudinally along the distribution manifold 238. In addition, a single supply channel 256 extends transversely across the 5 substantially parallel, equispaced channels at about a midpoint of the channels. The supply channels 256 facilitate distributing a fluid supplied by the inlet channel 254 across an area of the distribution manifold 238.

Each of the substantially parallel, equispaced supply channels 256 are coupled in flow communication to a plurality of resistance channels 257. The resistance channels 257 extend away from the supply channels 256 and are equispaced along the longitudinal length of the channels. In addition, the resistance channels 257 are formed symmetrically with each other along an axis of the respective supply channel 256. The resistance channels 257 have a size that is smaller than a size of the supply channels 256. Moreover, the resistance channels 257 are formed to create a tortuous flow path for the fluid, thereby facilitating an increase of the resistance of the fluid distribution network 244 to the flow of the fluid. Each one of the resistance channels 257 are coupled in flow communication to an outlet channel 258. Each outlet channel 258 is aligned with a respective protrusion member 234 for distributing the fluid through the fluidic passage 246 (FIG. 19A). In other embodiments, the channels 254, 256, 257, 258 may be formed in any configuration that enables the distribution manifold 238 to function as described herein.

### B. Plenum Assembly

In some embodiments, the device may comprise a plenum assembly slidably coupled to the fluidic block and configured to hold the fluidic distribution assembly.

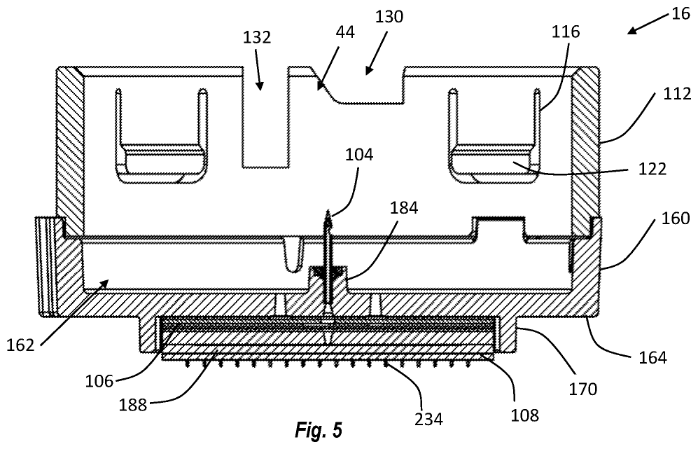

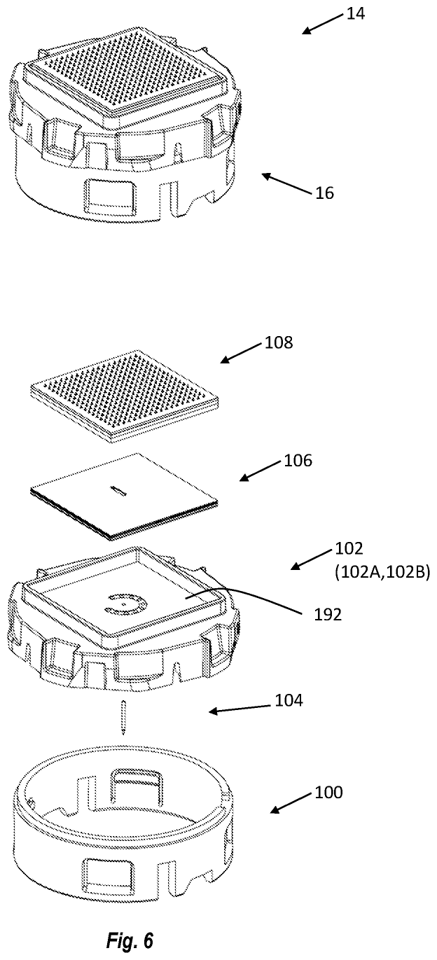

FIG. 5 is a sectional view of the plenum assembly 16 of the fluid delivery apparatus 10 in an active configuration according to an embodiment of the present disclosure. FIG. 6 is an exploded, perspective view of the plenum assembly 16 of FIG. 0.5. In the exemplary embodiment, the plenum assembly 16 includes a sleeve component 100, a plenum component 102 (A,B), a cannula 104, a plenum cap assembly 106 (broadly, “a gas extraction device”), and a fluidic delivery assembly 108 coupled together to form the unitary plenum assembly 16. In particular, the sleeve component 100 is coupled to the plenum component 102 to define a cavity 110 therein. In the exemplary embodiment, the sleeve component 100 is coupled to the plenum component 102 for example, and without limitation, via an adhesive bond, a weld joint (e.g., spin welding, ultrasonic welding, laser welding, or heat staking), and the like. Alternatively, the sleeve component 100 and the plenum component 102 may be coupled together using any connection technique that enables the formation of the plenum assembly 16.

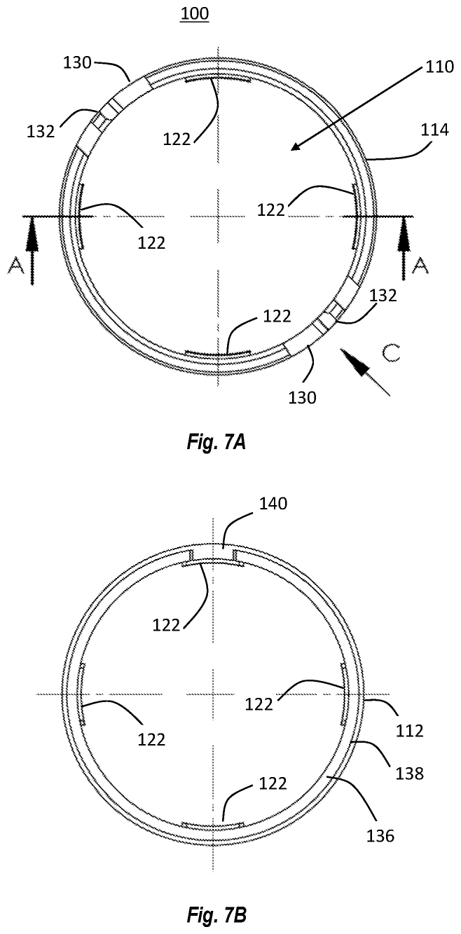

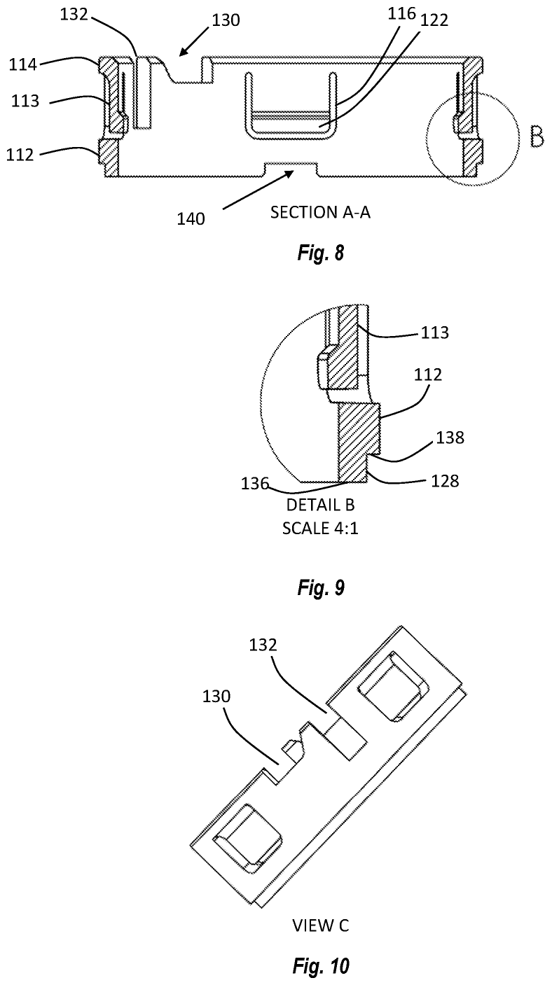

FIGS. 7A-7B are top and bottom views of a sleeve component of the plenum assembly. FIG. 8 is a section view of the sleeve component taken about line A-A shown in FIG. 7A. FIG. 9 is an enlarged view of section B shown in FIG. 9. FIG. 10 is a side view of the sleeve component from direction C shown in FIG. 7A.

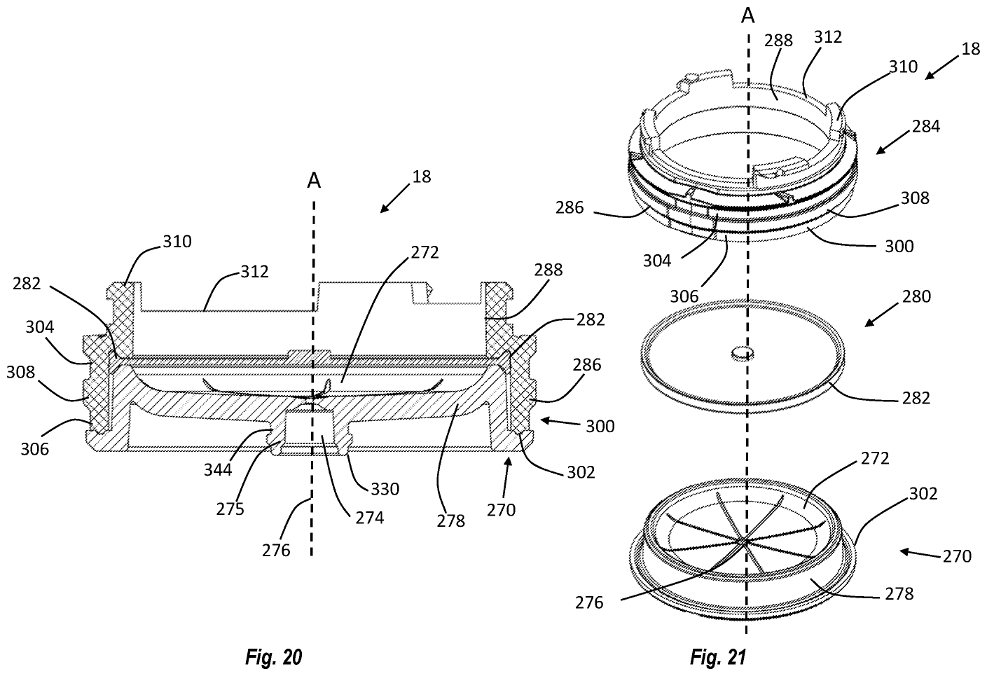

As illustrated in FIGS. 7A-10, in the exemplary embodiment, the sleeve component 100 includes a lower annular wall portion 112 and an upper annular wall portion 114. The upper annular wall portion 114 includes a plurality of flexible tabs 116 that extend substantially axially about the central axis of the sleeve component 100 and are formed integrally with the upper wall portion 114. The plurality of flexible tabs 116 are positioned equidistant about the central axis with respect to each other. In the exemplary embodiment, each flexible tab 116 includes a radially inward extending protrusion 122 that is positioned to engage the upper groove 304 of the outer wall 208 of the cartridge assembly 18 (as shown in FIG. 20) to facilitate properly positioning the cartridge assembly 18 in the non-activated and activated configurations. As shown in FIG. 9, the surface 136 or 138 provides a bonding surface for permanent bonding.

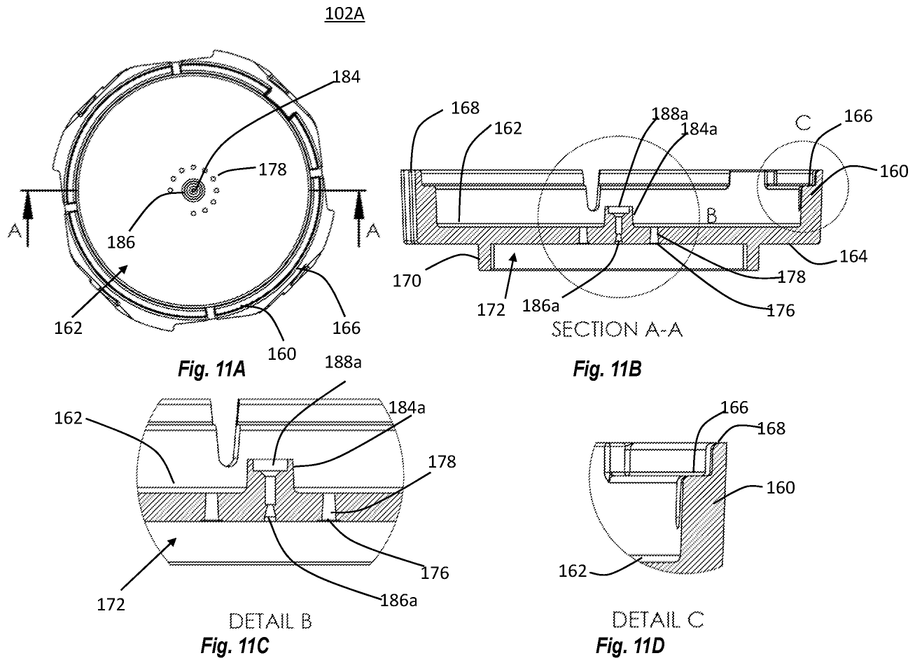

As shown in FIG. 10, the opening 132 is formed on the upper surface of the sleeve component and provides guide mechanism along with the protrusion member 372 of the insert of the mechanically controller assembly when the insert and the plenum assembly are engaged. FIGS. 11A-11D show an embodiment of a plenum component of the plenum assembly.

FIG. 11A is a top view of a plenum component 102A of the plenum assembly; FIG. 11B is a section view of the plenum component 102A taken about line A-A shown in FIG. 11A; FIG. 11C is an exploded view of section B shown in FIG. 11B; FIG. 11D is an exploded view of section C shown in FIG. 11B.

In the exemplary embodiment, the plenum component 102A includes a generally planar annular disk body portion 160 that extends horizontally across the lower wall portion 112 of the sleeve component 100 adjacent the bottom surface 136 to define the cavity 110. The body includes an upper surface 162 (FIGS. 11A-11B) and an opposite lower surface 164 (FIG. 11B).

Referring to FIGS. 11B and 11D, the plenum component 102A includes a step defines an inner horizontal surface 166 configured to engage with the sleeve component of the plenum assembly to facilitate properly positioning the plenum assembly 16 above a user's skin surface prior to use of the fluid delivery apparatus 10.

The sleeve component 100 is coupled to the plenum component 102 for example, and without limitation, via an adhesive bond, a weld joint (e.g., spin welding, ultrasonic welding, laser welding, or heat staking), and the like.

As shown in FIG. 11C, a mount 184 extends upwardly from the upper surface 162 of the plenum component 102 and a cannular 104 is coupled to the mount 184 and in fluid communication to a fluid passage 186 that extends through the plenum component 102. As shown in FIG. 2B, the cannula 104 is coupled to the plenum component 102 via an interference fit with the mount 184 and an adhesive disposed in a cavity 188 defined in the mount 184. As used herein, the phrase “interference fit” means a value of tightness between the cannula 104 and the mount 184, i.e., an amount of radial clearance between the components. A negative amount of clearance is commonly referred to as a press fit, where the magnitude of interference determines whether the fit is a light interference fit or interference fit. A small amount of positive clearance is referred to as a loose or sliding fit. Alternatively, the cannula 104 may be coupled to the mount 184 using any suitable fastening technique that enables the plenum component 102 to function as described herein. In the exemplary embodiment, an upper portion the cannula 104 is sharply pointed and extends upwardly away from the plenum component 102, such that the cannula 104 can pierce a portion of the cartridge assembly 18, as is described herein.

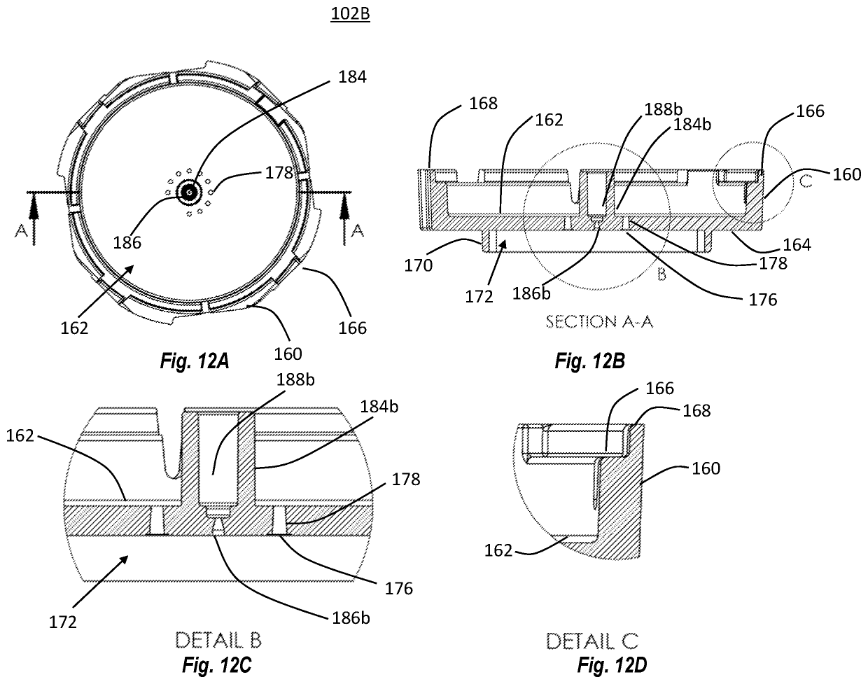

FIGS. 12A-12D show another embodiment of a plenum component of a fluid delivery apparatus according to an embodiment of the present disclosure, which includes tubing system connected to an external pump. FIG. 12A is a top view of a plenum component of the plenum assembly; FIG. 12B is a section view of the plenum component taken about line A-A shown in FIG. 12A; FIG. 12C is an exploded view of section B shown in FIG. 12B; FIG. 12D is an exploded view of section C shown in FIG. 12B. The structure of the plenum component 102B as shown in FIGS. 12A-12D are substantially the same as the plenum component 102A shown in FIGS. 11A-11D. In the embodiment of FIGS. 12-12D, it differs in that the cavity 188b is substantially cylindrical and configured to receive the tubing connected to the external pump whereas the cavity 188a as shown in FIG. 12C is sized to be coupled to the plenum component 102 via an interference fit with the mount 184.

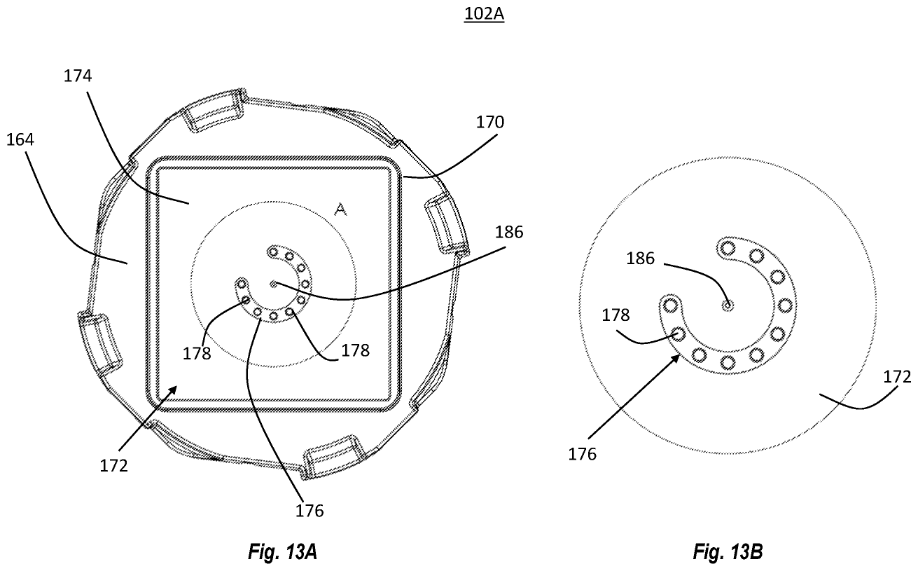

The lower surface 164 of the plenum component 102 (FIG. 13A) includes a rectangular frame portion 170 that extends downwardly from the body portion 160. The frame portion 170 defines a mounting space 172 for coupling the plenum cap assembly 106 and the fluidic distribution assembly 108 to a mounting surface 174 located within the mounting space 172.

The plenum component 102 includes an arcuate channel 176 having a plurality of axially extending apertures 178 defined therein. As best illustrated in FIGS. 13A-13B, the arcuate channel 176 is defined in the mounting surface 174 within the mounting space 172. The arcuate channel 176 has a predetermined width that is centered about a center radius concentric with the central axis of the plenum component 102. In the exemplary embodiment, the arcuate channel 176 extends circumferentially about 270°. In other embodiments, the arcuate channel 176 can extend any circumferential angle that enables the plenum component 102 to function as described herein. In the exemplary embodiment, the axially extending apertures 178 are uniformly disposed in the arcuate channel 176. Each aperture 178 is centered on the center radius and extends through the body portion 160 from the lower surface 164 to the upper surface 162. In the exemplary embodiment, the plenum component 102 includes ten axially extending apertures 178. Alternatively, in other suitable embodiments, the plenum component 102 can include any number of axially extending apertures 178 that enables the plenum component 102 to function as described herein.

Plenum Cap Sub-Assembly

In some embodiments, the plenum assembly may comprise a plenum cap sub-assembly. The plenum cap sub-assembly may be configured to facilitate gas extraction from the fluid. The plenum cap assembly may permit venting of air from the fluidic pathway. The plenum cap sub-assembly may comprise a plenum vent gasket comprising a plurality of layers (e.g., five layers) including adhesive layer, a vent membrane, and an impermeable membrane.

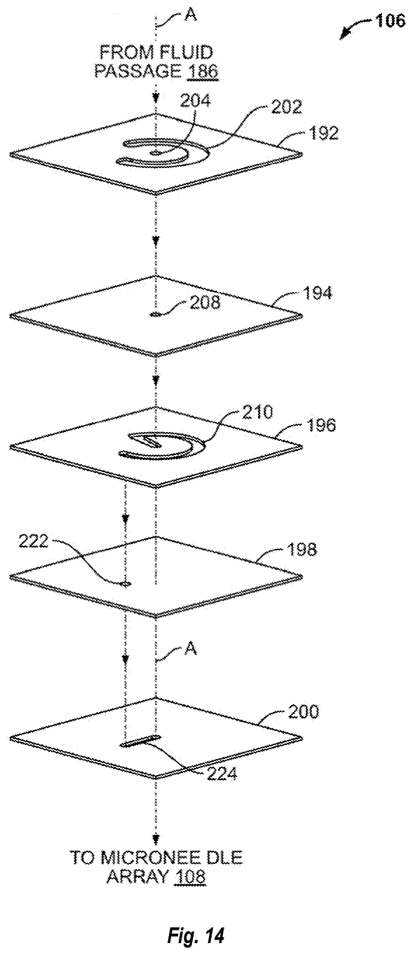

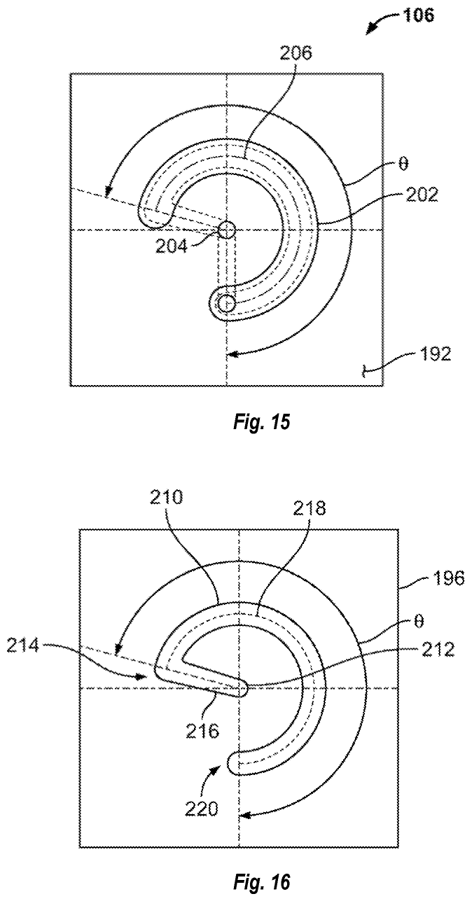

FIG. 14 is an exploded, schematic of the plenum cap assembly 106 of the fluid delivery apparatus 10 shown in FIG. 1A. FIG. 15 is a top view of the plenum cap assembly 106. In the exemplary embodiment, the plenum cap assembly 106 is a unitary assembly comprising a plurality of layers bonded together. The plenum cap assembly 106 is bonded to the mounting surface 174 of the plenum component 102 via a first adhesive layer 192, which is fabricated from pressure-sensitive adhesive film. The first adhesive layer 192 includes an arcuate slot 202 defined therethrough. The arcuate slot 202 is positioned substantially concentric with an aperture 204 formed coaxial with the central axis “A.” The arcuate slot 202 has a predetermined width that is centered about a center radius 206. The center radius 206 is concentric with the central axis “A.” In the exemplary embodiment, the arcuate slot 202 extends circumferentially at an angle θ. In other embodiments, the arcuate slot 202 can extend any circumferential angle θ that enables the plenum cap assembly 106 to function as described herein. In the exemplary embodiment, the arcuate slot 202 is configured to at least partially correspond to the arcuate channel 176 of the plenum component 102 and the aperture 204 is positioned to correspond to the fluid passage 186.

The plenum cap assembly 106 includes a vent membrane 194 coupled to the first adhesive layer 192 opposite the plenum component 102. In one embodiment, the vent membrane 194 includes a fluid inlet aperture 208 formed coaxial with the central axis “A.” In the exemplary embodiment, the aperture 208 is substantially the same size as the aperture 204 of the first adhesive layer 192. In one suitable embodiment, the vent membrane 194 is fabricated from a gas permeable oleophobic/hydrophobic material. It is understood that other types of suitable materials can be used in other embodiments. For example, and without limitation, in one embodiment, the vent membrane 194 is fabricated from an acrylic copolymer membrane formed on a nylon support material, such as Versapor®-200R (Pall Corporation, NY). In the exemplary embodiment, the pore size of vent membrane 194 is about 0.2 microns. The vent membrane 194 has a flow rate for air in the range between about 200 milliliters/minute/centimeter² (mL/min/cm²) and about 2000 mL/min/cm²), as measured at about 150 kilopascal (kPa). In addition, the vent membrane 194 has a minimum fluid bubble pressure in the range between about 35 kilopascal (kPa) and about 300 kPa. In one suitable embodiment, the vent membrane 194 has a flow rate for air of at least 250 mL/min/cm², as measured at about 150 kPa, and a minimum fluid bubble pressure of at least 150 kPa. Alternatively, the vent membrane 194 can be fabricated from any gas permeable material that enables the plenum cap assembly 106 to function as described herein.

FIG. 16 is a top view of a second adhesive layer 196 of the plenum cap assembly 106. In the exemplary embodiment, the second adhesive layer 196 is formed from a pressure-sensitive adhesive film and is coupled to the vent membrane 194 opposite the first adhesive layer 192. The second adhesive layer 196 is formed similarly to the first adhesive layer 192 and includes an arcuate slot 210 defined therethrough. The arcuate slot 210 is configured to form a tortuous flow path that extends generally perpendicular to the central axis “A” to facilitate removing gas from the fluid. The arcuate slot 210 is sized and positioned to substantially correspond to the slot 202 of the first adhesive layer 192. The slot 210 is positioned concentric with a central aperture portion 212, which is formed coaxial with the central axis “A.” A first end 214 of the arcuate slot 210 is connected to the central aperture portion 212 with a linear slot portion 216. The arcuate slot 210 has a predetermined width that is centered about a center radius 218, which corresponds to the center radius 206 of the first adhesive layer 192. In the exemplary embodiment, the arcuate slot 210 extends circumferentially at the same angle θ as the arcuate slot 202. In other embodiments, the arcuate slot 210 can extend any circumferential angle that enables the plenum cap assembly 106 to function as described herein.

The plenum cap assembly 106 includes an impermeable membrane 198 coupled to the second adhesive layer 196 opposite the vent membrane 194. In the exemplary embodiment, the impermeable membrane 198 includes a fluid aperture 222 formed coaxial with a second end 220 of the arcuate slot 210. In the exemplary embodiment, the aperture 222 is substantially the same size as the apertures 204, 208 of the first adhesive layer 192 and the vent membrane 194, respectively. The impermeable membrane 198 is fabricated from a gas and liquid impermeable material. For example, and without limitation, in one embodiment, the impermeable membrane 198 is fabricated from a polyethylene terephthalate (PET) film. Alternatively, the impermeable membrane 198 can be fabricated from any gas and liquid impermeable material that enables the plenum cap assembly 106 to function as described herein.

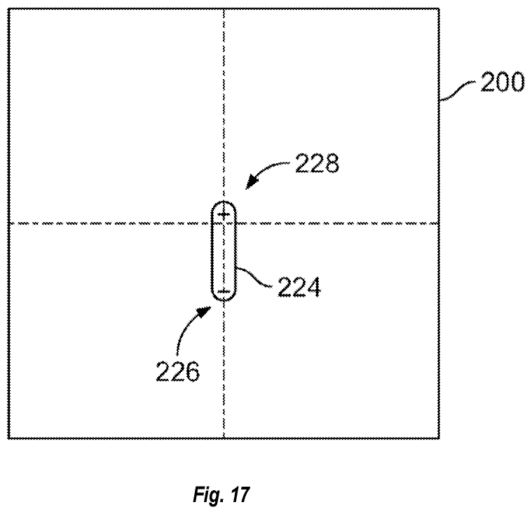

FIG. 17 is a top view of a third adhesive layer 200 of the plenum cap assembly 106. In the exemplary embodiment, the third adhesive layer 200 is formed from a pressure-sensitive adhesive film and is coupled to the impermeable membrane 198 opposite the second adhesive layer 196. The third adhesive layer 200 includes a slot 224 defined therethrough. The slot 224 includes a first end 226 that is sized and positioned to substantially correspond to the aperture 222 of the impermeable membrane 198. In addition the slot extends from the first end 226 to a second end 228, which includes a full radius end sized substantially similar to the apertures 204, 208 of the first adhesive layer 192 and the vent membrane 194, respectively. Moreover, the second end 228 is positioned substantially coaxial with the central axis “A.”

### C. Collet Assembly

In some embodiments, the device may comprise a collet assembly constituting the housing of the device and configured to contact a surface of the subject's skin sufficient for penetration of the plurality of protrusions into the surface of the subject's skin and across the dermal barrier. In some embodiments, the collet assembly comprises a collet and a collet lock. The collet and the collet lock may be coupled together using any connection technique that enables the formation of the collet assembly.

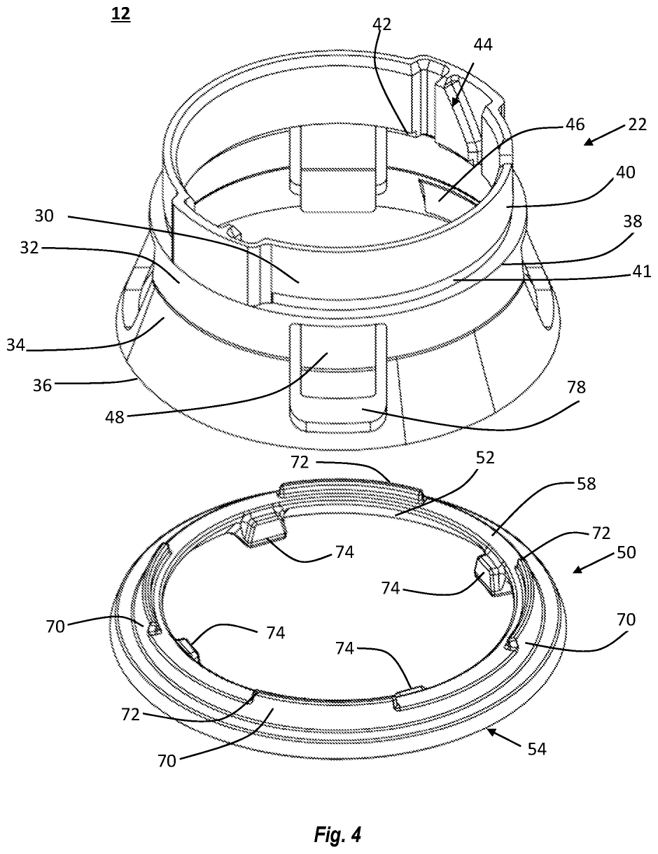

FIG. 4 is an exploded, perspective of the collet assembly 12 of the fluid delivery apparatus 10 according to an embodiment of the present disclosure. Referring to FIG. 4, the collet assembly includes a collet 22 coupled to a collet lock 50. In the embodiment shown in FIG. 4, the collet 22 is formed in a generally frustoconical shape, having a hollow interior space defined therein. The collet 22 is coupled to the collet lock 50 to form a unitary assembly (shown in FIG. 1).

An upper rim of the collet 22 defines an opening to the interior space. A cylindrical upper wall 30 extends generally vertically downward from the upper rim towards a central portion 32 of the collet 22. A lower wall 34 extends downward at an outward angle from the central portion 32 toward a base 36 (or lower edge) of the collet 22. The upper wall 30, central portion 32, and the lower wall 34 collectively define the interior space 24.

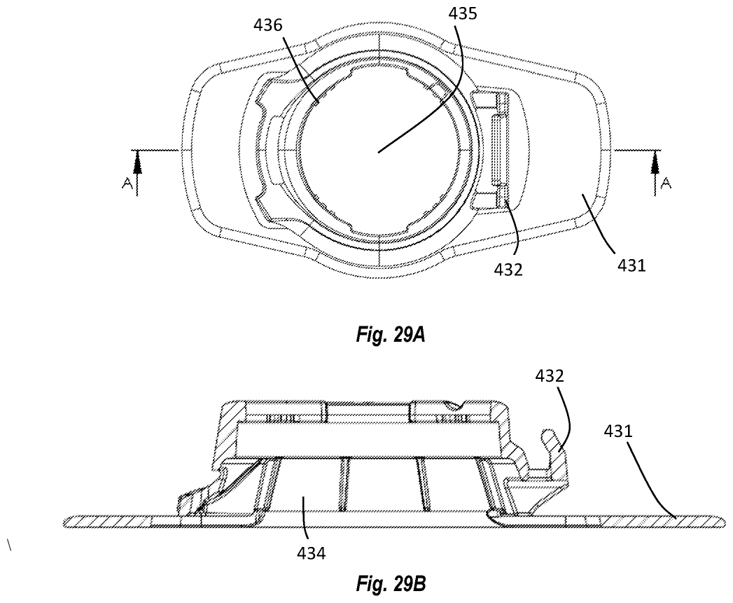

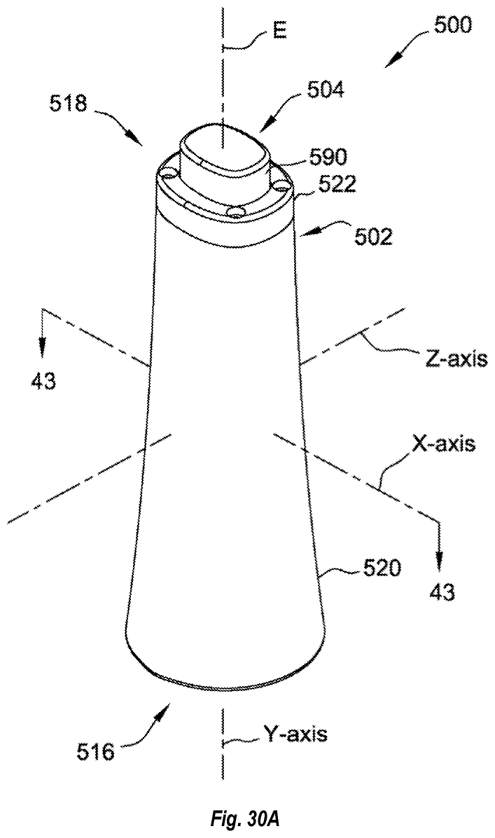

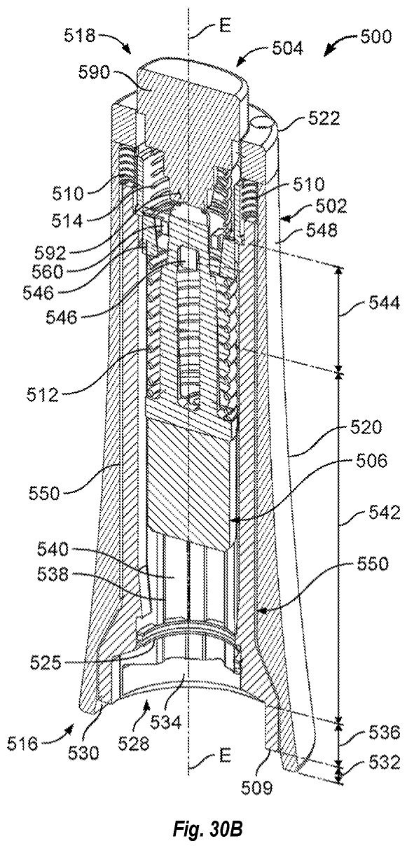

A step 38 extends around the upper wall 30, defining a recessed portion 41 extending upwardly from the outer horizontal surface 40 (or ledge) and configured to engage an attachment band (shown in FIGS. 29A), as is described further herein. The step 38 also defines an inner horizontal surface 42 (or step) configured to engage with the plenum assembly 16 to facilitate properly positioning the plenum assembly 16 above a user's skin surface prior to use of the fluid delivery apparatus 10.

In addition, the collet 22 includes one or more stops 46 configured to facilitate positioning of the collet lock 50 when coupled to the collet 22. For example, and without limitation, the one or more stops 46 are formed as inward extending projections formed on lower wall 34. The stops 46 can have form or shape that enables the stops 46 to function as described herein.

As illustrated in FIG. 4, the collet 22 includes a plurality of flexible tabs 48 formed integrally with the upper wall 30 and positioned about and equidistant from the central axis. In particular, the plurality of flexible tabs 48 includes a free end 78 that angles radially inward and is configured to engage with the plenum assembly 16 to facilitate properly positioning the plenum assembly 16 at the user's skin surface during use of the fluid delivery apparatus 10.

As illustrated in FIG. 4, in the exemplary embodiment, the collet lock 50 is generally ring-shaped, having a convex inner surface 52 extending from a lower outer edge 54 of the collet lock 50 to a generally cylindrical inner wall. The inner wall extends upward to an upper surface 58. The collet lock 50 includes a generally cylindrical outer wall that is concentric with inner wall and extends upward from the lower outer edge 54.

In the exemplary embodiment, the outer wall of the collet lock 50 includes an upper outer surface 70 that inclines inward at an angle substantially parallel to the lower wall 34 of the collet to facilitate face-to-face engagement therewith. In addition, the upper surface 58 includes a plurality of stop members 72 that extend upward and are configured to engage the one or more stops 46 of the collet 22 to facilitate properly positioning of the collet lock 50 when coupled to the collet 22. Extending radially inward from the convex inner surface 52 is a plurality of tabs 74 configured to engage with the plenum assembly 16 to facilitate properly positioning the plenum assembly 16 at the user's skin surface during use of the fluid delivery apparatus 10.

### D. Cartridge Assembly

In some embodiments, the device may comprise a cartridge assembly. The cartridge assembly may comprise a reservoir component including an upper cavity and an opposing lower cavity coupled together in flow communication with the fluidic block via the cannula of the plenum assembly.

FIG. 20 is a sectional view of the cartridge assembly 18 of the fluid delivery apparatus 10 shown in FIG. 1. FIG. 21 is an exploded, schematic of the cartridge assembly 18. The cartridge assembly 18 includes a reservoir component 270 formed generally concentric about the central axis “A.” The reservoir component 270 includes an upper cavity 272 and an opposing lower cavity 274 coupled together in flow communication via a fluid passage 276. In the exemplary embodiment, the upper cavity 272 has a generally concave cross-sectional shape, defined by a generally concave body portion 278 of the reservoir component 270.

The lower cavity 274 has a generally rectangular cross-sectional shape, defined by a lower wall 275 that extends generally vertically downward from a central portion of the concave body portion 278. An upper portion of the end of the fluid passage 276 is open at the lowest point of the upper cavity 272, and an opposite lower portion of the fluid passage 276 is open at a central portion of the lower cavity 274. The lower portion of the fluid passage 276 expands outward at the lower cavity 274, forming a generally inverse funnel cross-sectional shape. In other embodiments, the cross-sectional shapes of the upper cavity 272, the lower cavity 274, and the fluid passage 276 may be formed in any configuration that enables the reservoir component 270 to function as describe herein.

The cartridge assembly 18 also includes an upper sealing member 280 (or membrane) configured to couple to the reservoir component 270 and close the upper cavity 272. The upper sealing member 280 is formed as an annular sealing membrane and includes a peripheral ridge member 282 to facilitate sealingly securing the upper sealing member 280 to the cartridge assembly 18. A cartridge housing 284 extends over the upper sealing member 280 and is configured to fixedly engage the reservoir component 270. This facilitates securing the upper sealing member 280 in sealing contact with the reservoir component 270, thereby closing the upper cavity 272.

In the exemplary embodiment, the cartridge housing 284 includes an annular, vertically-extending wall 286 that has an inward extending flange member 288 configured to couple to the peripheral ridge member 282 of the upper sealing member 280. In particular, the flange member 288 cooperates with the concave body portion 278 of the reservoir component 270 to compress and sealingly secure the upper sealing member 280 therebetween. In the exemplary embodiment, a lower end 300 of the vertically-extending wall 286 is coupled to a flange 302 of the reservoir component 270 via welding, for example, and without limitation, ultrasonic welding, spin welding, laser welding, and/or heat staking. In other embodiments, the vertically-extending wall 286 may be coupled to a flange 302 using any connection technique that enables the cartridge housing 284 to fixedly engage the reservoir component 270, for example, and without limitation, via an adhesive bond and the like.

The cartridge housing 284 also includes an upper groove 304 and a lower groove 306 formed circumferentially in an outer surface 308 of the vertically-extending wall 286. The upper and lower grooves 304, 306 are sized and shaped to engage the plurality of flexible tabs 116 of the sleeve component 100, and, in particular, the radially inward extending protrusions 122 formed at the free second end of the plurality of flexible tabs 116, as is described herein. In addition, the cartridge housing 284 also includes a plurality of protrusion members 310 formed on an upper edge portion 312 of the vertically-extending wall 286 and configured to couple to the mechanical controller assembly 20 to secure it to the cartridge assembly 18, as described herein.

### E. Cap Sub-Assembly

In some embodiments, the device may comprise a cap assembly. The cap assembly may comprise a septum component configured to couple to the reservoir component and close the lower cavity of the cartridge assembly. The cap assembly may further comprise a snap cap configured to facilitate access to the septum component during use of the fluid delivery apparatus.

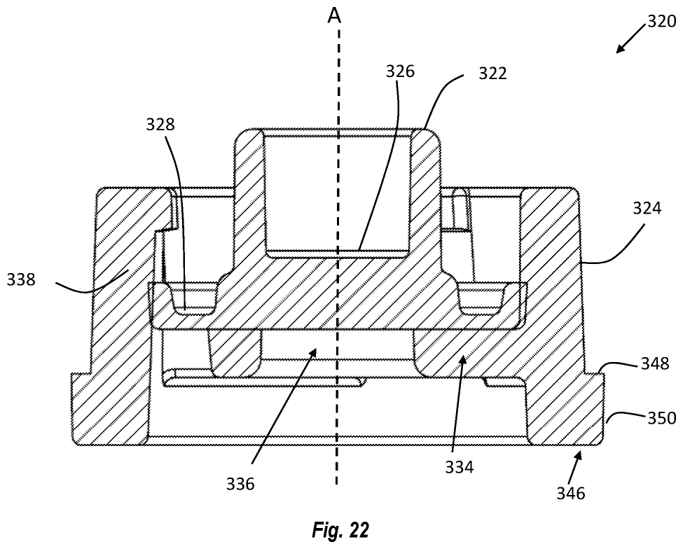

FIG. 22 is a sectional view of the cap assembly 320 of the fluid delivery apparatus 10 shown in FIG. 1A. In the exemplary embodiment, the cap assembly 320 includes a septum component 322 and a snap cap component 324 coupled together. The septum component 322 is configured to couple to the reservoir component 270 and close the lower cavity 274 of the cartridge assembly 18. The septum component 322 has a lower wall 326 that extends substantially perpendicular to the central axis “A.” The lower wall 326 includes a peripheral channel 328 that is configured to sealingly engage a rim 330 of the lower wall 275 of the reservoir component 270. The septum component 322 also includes an annular upper seal wall, transverse to the lower wall 326, and that extends axially into the lower cavity 274 when coupled to the reservoir component 270. The snap cap component 324 extends over the septum component 322 and is configured to fixedly engage the lower wall 275 of the reservoir component 270. This facilitates securing the septum component 322 in sealing contact with the reservoir component 270, thereby sealingly closing the lower cavity 274.

The snap cap component 324 includes a lower wall 334 that has a central opening 336 to facilitate access to the lower wall 326 of the septum component 322 during use of the fluid delivery apparatus 10. The snap cap component 324 includes an annular vertically-extending wall 338 that extends upwardly and downwardly from a periphery of the lower wall 334. The vertically-extending wall 338 may engage the lower wall 275 of the reservoir component 270 using any connection technique that enables the snap cap component 324 to fixedly engage the lower wall 275, for example, and without limitation, via an interference fit, an adhesive bond, a weld joint (e.g., spin welding, ultrasonic welding, laser welding, or heat staking), and the like. In the exemplary embodiment, a lower portion 346 includes an outwardly extending flange portion 348 that defines a peripheral sealing surface 350 configured to engage an additional seal member (not shown) that extends between the snap cap component 324 and the upper rim 168 of the annular central wall of the plenum component 102.

### F. Controller Assembly

If desired, the rate of delivery of the fluidic composition may be variably controlled by the pressure-generating means. In some embodiments, the device may comprise a controller assembly slidably coupled to the fluidic block and configured to control the flow of the fluidic composition during delivery of the fluidic composition through the plurality of protrusions.

Desired delivery rates as used herein may be initiated by driving the fluidic composition described herein with the application of pressure or other driving means, including pumps, syringes, pens, elastomer membranes, gas pressure, piezoelectric, electromotive, electromagnetic or osmotic pumping, or use of rate control membranes or combinations thereof.

In some embodiments, the controller assembly includes an external infusion pump and a tubing system to provide a pressured flow of the fluidic composition from a reservoir for holding the fluidic composition located exterior of the device into the device and through the plenum to the fluidic block. Any known infusion pumps that are capable of delivering fluids in predetermined amounts may be used. In some embodiments, the external infusion pump is a syringe pump, an elastomeric pump, or a peristaltic pump. In some embodiments, the external infusion pump is a portable.

Mechanical Controller Assembly

In some embodiments, the controller assembly comprises a mechanical controller assembly. The mechanical controller assembly may comprise a controller housing, a pushing component that may be in the form of a plunger member or the like, positionable in a range from a first position proximal to the plenum, to a second position distant from the plenum; and a biasing assembly comprising at least one biasing member positioned between the controller housing and the plunger for moving the plunger relative to the controller housing. The biasing member is configured to apply a pressure to the plunger in an axial direction away from the controller housing, wherein the pressure applied to the plunger member by the biasing assembly is transmitted to the plenum and facilitates displacing the fluidic composition into the fluidic block.

The biasing member may comprise one or more of springs, and/or one or more other suitable force providing features that may be in the form of elastic objects. In some embodiments, the first force provider or spring is larger than, and may be stronger than, the second force provider or spring.

In some embodiments, the controller housing may comprise a terminal portion that may be in the form of a plate or disk. The terminal portion or disk may be generally or at least somewhat dome-shaped and may serve as a push-button or portion of a push-button for being manually pressed. In some embodiments, the controller housing as a whole, or portions thereof, may be referred to as a push-button.

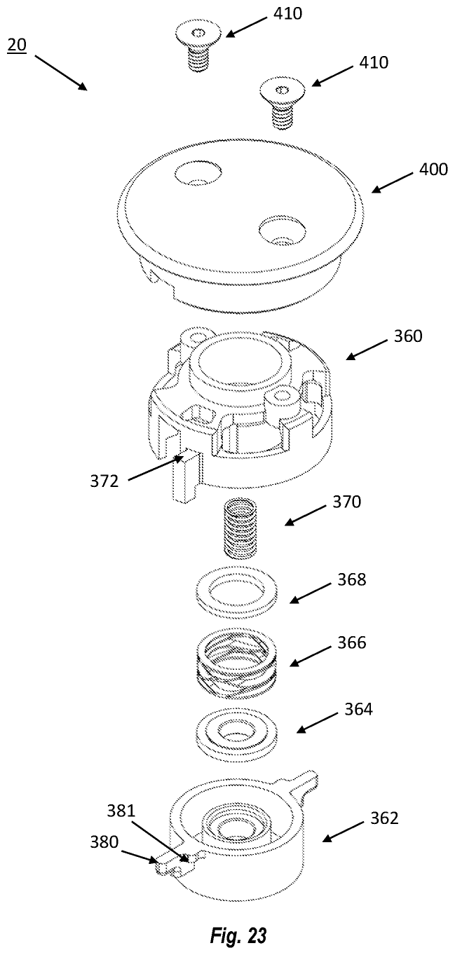

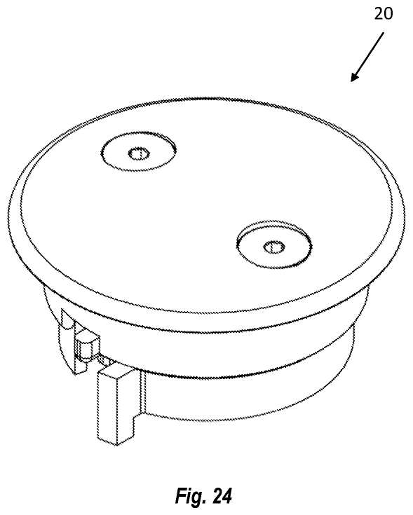

FIG. 23 is an exploded, perspective view of the mechanical controller assembly 20 of the fluid delivery apparatus 10 shown in FIG. 1A. FIG. 24 shows the assembled mechanical controller assembly 20 shown in FIG. 23. The mechanical controller assembly 20 includes at least a controller housing, a plunger component 362, and a biasing assembly positioned between the controller housing and the plunger component for biasing the plunger component in an axial direction away from the body component.

The biasing assembly includes at least one biasing member. In one embodiment, at least one biasing member may include any biasing component that enables the biasing assembly to function as described herein, including, for example, elastic (spring), resilient materials; foams; fluid (gas or liquid) compression members, and the like. In some embodiment, each biasing member has a different length and a different force constant (or force profile). The biasing assembly also includes an insert component. In the exemplary embodiment, each biasing member has a different diameter.

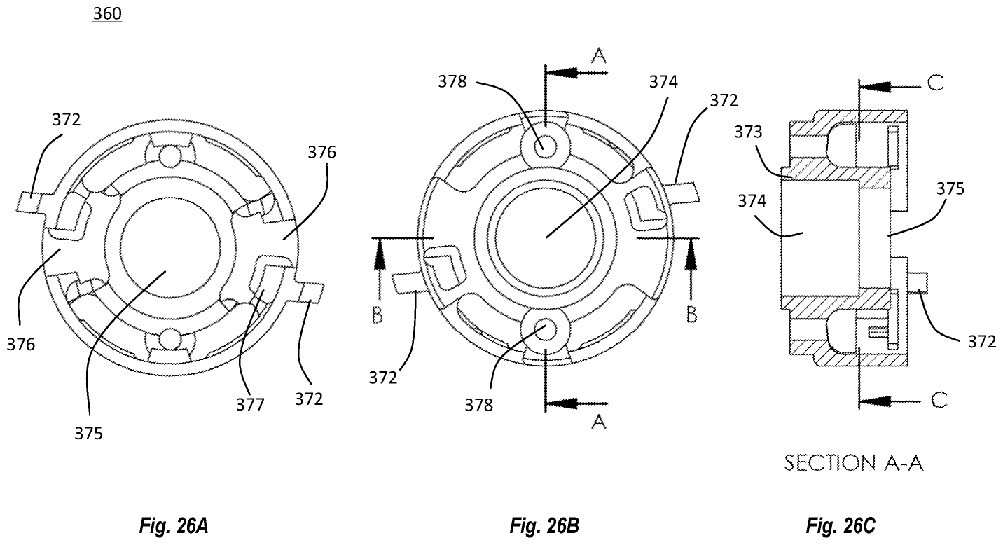

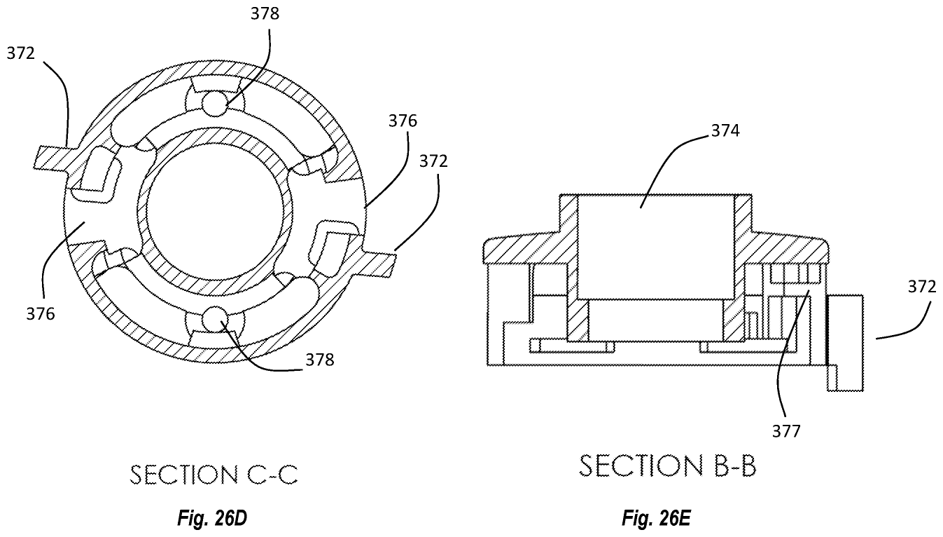

As illustrated in the FIGS. 23, 26A-26C, the first and second biasing members 366, 370 positioned in the cylindrical-shaped inner portion 374 (FIG. 26B) of the insert component 362. The first biasing member 366 extends from the cylindrical-shaped inner portion of the insert component 360 to the cylindrical-shaped inner portion 384 of the plunger component 362.

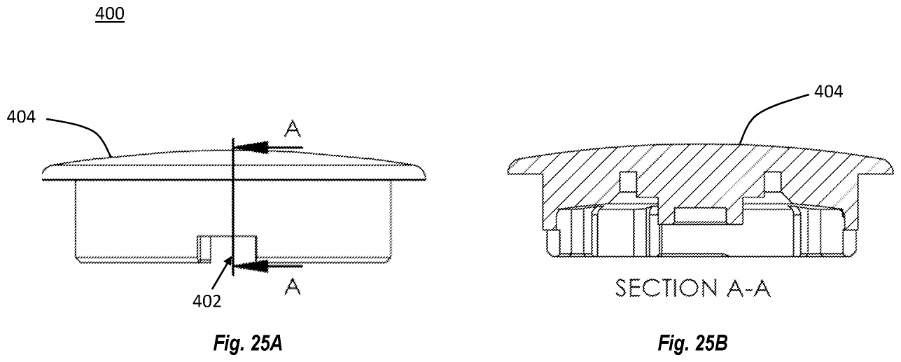

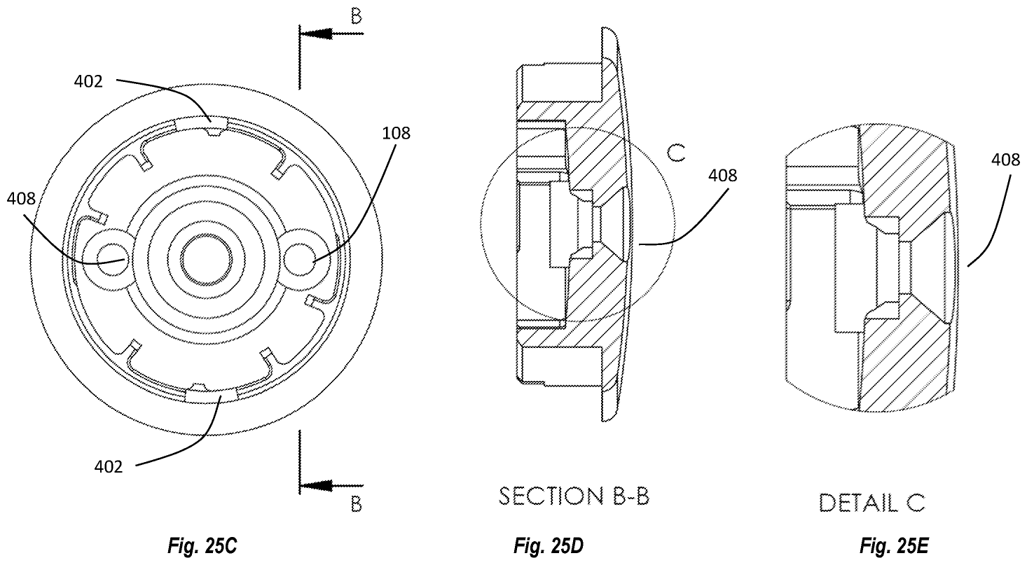

In some embodiments, the controller housing includes a housing component 400. FIG. 25A is a side view of the housing component 400. FIG. 25B is a sectional view of the housing component taken about line A-A of FIG. 25A. FIG. 25C is a bottom view of the housing component 400. FIG. 25D is a sectional view of the insert component 360 taken about line B-B of FIG. 25C. FIG. 25E is an enlarged view of the insert component 360 from FIG. 25E. In the exemplary embodiment, the housing component 400 comprises a generally dome-shaped terminal portion (button) 404 and an annular sidewall 401 with a pair of cutouts 402 opposite to each other. As illustrated in FIGS. 25A-25C, the annular sidewall 401 includes cutouts 402 to enable the lever components 380 of the plunger component 362 to extend therethrough. The terminal portion 404 includes a pair of threaded holes 408. The threaded holes 408 receive mechanical hardware 410 used to couple the housing component 400 to the insert component 360.

In some embodiments, the controller housing comprises an insert component. FIGS. 26A-26E show top and bottom views of the insert component; and sectional views of the insert component take about line A-A, B-B, and C-C, respectively.

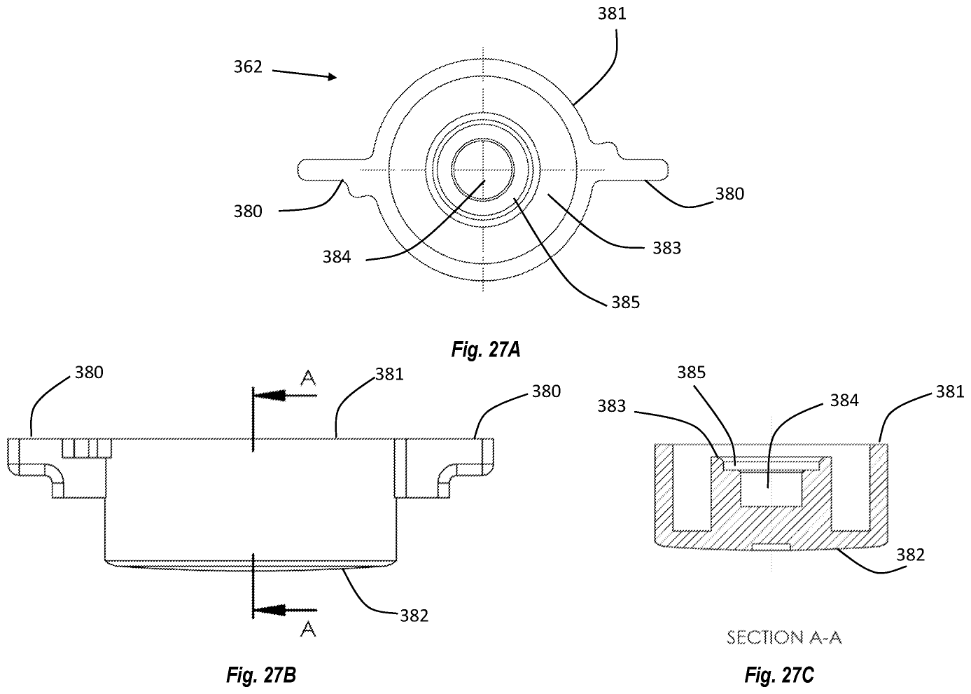

In some embodiments, the mechanical controller assembly comprises a plunger component. FIGS. 27A-27C show top and side views of the plunger component; and sectional views of the plunger component take about line A-A, respectively. The plunger component includes a disk-shaped domed component 382 with an outer annular wall portion and an inner annular guide wall portion 383 coaxially extending vertically-upward from the domed component 382. As shown in FIG. 27C, the inner guide portion 384 of the plunger component 362 is configured to receive the second biasing member 370. In one embodiment, the plunger component 362 is configured to engage the upper sealing member of the cartridge assembly via force applied by the biasing assembly during use of the fluid delivery apparatus 10.

An embodiment of the mechanical controller assembly and its operation to control the rate of delivery of the fluidic composition through the fluidic distribution assembly is provided.

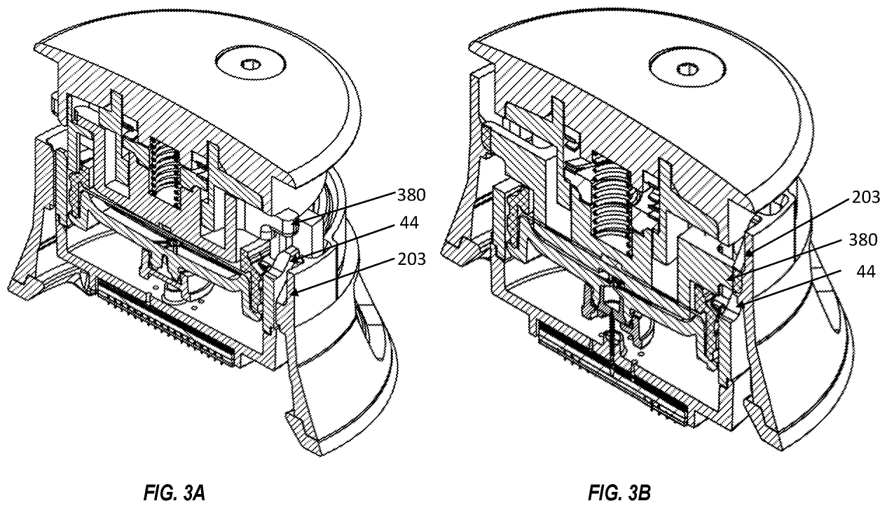

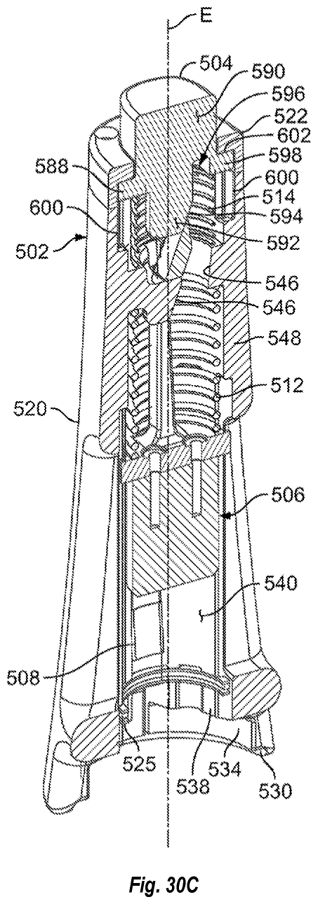

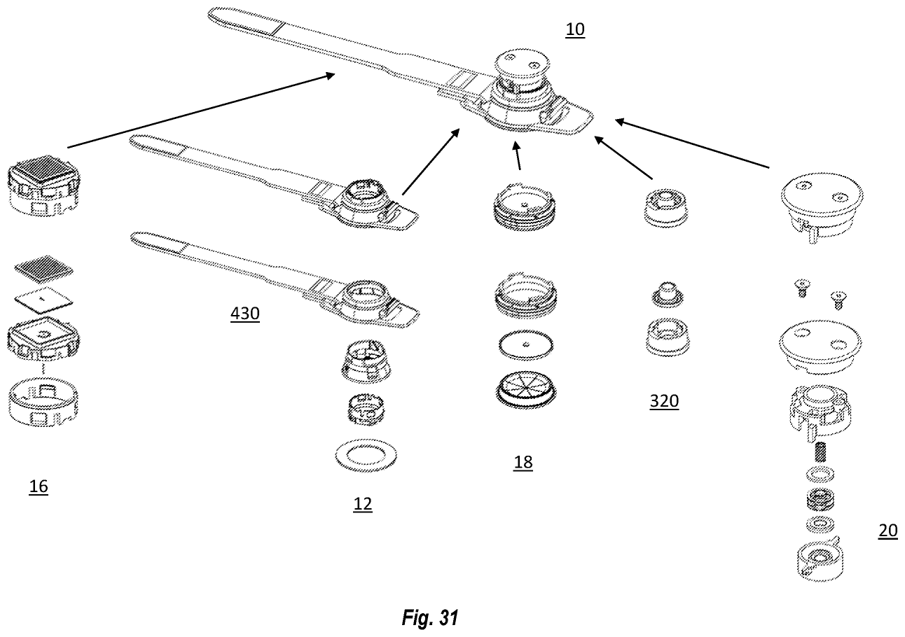

After the fluid delivery apparatus 10 is properly attached to the user and configured in the non-activated configuration shown in FIGS. 2A and 3A, the fluid delivery apparatus 10 can be activated by pressing the button (or terminal portion) 404 of the housing component 400 to release the plunger component 362. In one embodiment, a tool (an applicator 500 (as shown in FIGS. 30A-30C) or any other application device) configured to press the button 404 may be used. When the button 404 is pressed, the pivot about the cylindrical pins 452 such that the concave cutouts 458 of the latch portions pivot into axial alignment with the central axis “A.” This enables the plunger component 362 to disengage and contact the upper sealing member 280 of the cartridge assembly 18.

As shown in FIGS. 26A-26E, the lever component 380 of the plunger component 362 is configured to engage with protrusion member 377 from inner wall of the insert component 360 to facilitate retaining the plunger component 362 in a non-activated configuration. As best illustrated in FIGS. 3A-3B, upon application of the pressure, the lever component 380 of the plunger component 362 slides down on the angled surface 202 of the opening 130 of the sleeve component 100 of the plenum assembly 16. The opening 203 of the collet 22 provides clearance for the movement of the lever component 380. This enables the plunger component 362 to release from being retained by the insert component 360 and contact the upper sealing member 280 of the cartridge assembly 18.

As illustrated in FIG. 2B, the axial location of the upper ends of the second biasing member 370 and the first biasing member 366 are axially displaced with respect to each other. Further, as described herein, the second biasing member 370 and the first biasing member 366 have different lengths and force constants, thus the axial force applied to the plunger component 362 changes with respect to the displacement of the plunger component 362.

When the plunger component 362 is released, the first biasing member 366 and the second biasing member 370 apply force to the plunger component 362, i.e., a first force profile for the activated configuration of the fluidic delivery apparatus. As the plunger component 362 is displaced axially, the second biasing member 370 and the first biasing member 366 apply the force to the plunger component 362. As the plunger component 362 is displaced, the second biasing member 370 and the first biasing member 366 extend such that the force exerted on the plunger component 362 decreases. At a predetermined axial displacement of the plunger component 362, the first biasing member 366 becomes fully extended or is prevented from being extended further, e.g., by component 364 facing the surface 385 of the plunger component. At this position, the second biasing member 370 continues to apply a force to the plunger component 362, i.e., a second force profile for the activated configuration.

In some embodiments, the pressure applied to the plunger component 362 by the first and second biasing member 366, 370 is transmitted to the cartridge assembly 18. As illustrated in FIGS. 2A and 3A, in the non-activated configuration, the tip of the cannula 104 is within the cap assembly, but clear from the lower cavity 274 of the cartridge assembly 18. As illustrated in FIGS. 2B and 3B, in the activated configuration, the cannula 104 penetrates into the lower cavity 274 of the cartridge assembly 18. The pressure facilitates displacing the fluid contained in the upper cavity 272 through the cannula 104 and into the fluid passage 276. The fluid exits the fluid passage 276 by flowing into the plenum cap assembly 106. With reference to FIG. 14, the fluid flows downwardly through the aperture 204 of the first adhesive layer 192, the aperture 208 of the vent membrane 194, and into the arcuate slot 210 of the second adhesive layer 196. The impermeable membrane 198 is coupled to the bottom of the second adhesive layer 196, thereby preventing the fluid from passing directly therethrough. As such, the pressure applied by the biasing assembly forces the fluid to fill the arcuate slot 210, where it is channeled to the aperture 222 in the impermeable membrane 198. The fluid passes through the aperture 222 where it enters the slot 224 formed in the third adhesive layer 200. The fluid is channeled by the slot 224 to the inlet channel 254 of the fluid distribution assembly 108. The fluid is channeled to the inlet channel 254 of the fluid distribution assembly 108 and enters the distribution manifold 238, and then the fluid flows through the supply channels 256, the resistance channels 257, and the outlet channels 258 to the passageways 246 of the protrusions 234 and into the user's skin.

In some embodiments, the device is maintained at the activated configuration to deliver the fluidic composition to the target location (e.g., the lymphatic system of the subject) at a flow rate determined by the second force profile of the controller assembly. The flow rate of the fluidic composition may be maintained for at least a predetermined time period. In some embodiments, the flow rate of the fluidic composition does not change (i.e., is constant) for at least a predetermined time period. In some embodiments, the flow rate of the fluidic composition increases for a predetermined time period. In some embodiments, the flow rate of the fluidic composition decreases for at least a predetermined time period. In some embodiments, the flow rate changes over time in a sinusoidal, parabolic, triangular, or step-wise manner (i.e., a triangular, sinusoidal, parabolic, or step-wise flow rate profile).

### G. Attachment Band Assembly

In some embodiments, the device may further comprise an attachment band assembly. The attachment band assembly may be configured to couple to the collet assembly to facilitate contact with a surface of the subject's skin sufficient for penetration of the plurality of protrusions into the surface of the subject's skin and across the dermal barrier.

In some embodiments, the attachment band assembly may comprise an annular body configured to attach to the collet of the collet assembly; and an attachment band (or strap) removably engaged with the annular body. In some embodiments, the annular body comprise a wall defining a hollow inner space and a coupling member to engage with a corresponding coupling member of the collet. In some embodiments, the attachment band may comprise a hoop-and-loop type fastening strap such that in use, the strap is threaded through a portion of the annular body and folded back to tighten the strap around the skin of the subject. The attachment band may include, for example, but without limitation, an arm band, a leg band, a waist band, wrist band, and the like. In some embodiments, the attachment band includes an attachment member configured to couple to the coupling members of the collet.

The strap may extend generally radially outward from the annular body. In one embodiment, the strap has a width that is less than a diameter of the annular body. In one embodiment, the strap may have any width that enables the attachment band to function as described herein. In some embodiments, the annular body and the straps are fabricated separately and assembled using any fastening method that enables the attachment band to function as described herein.

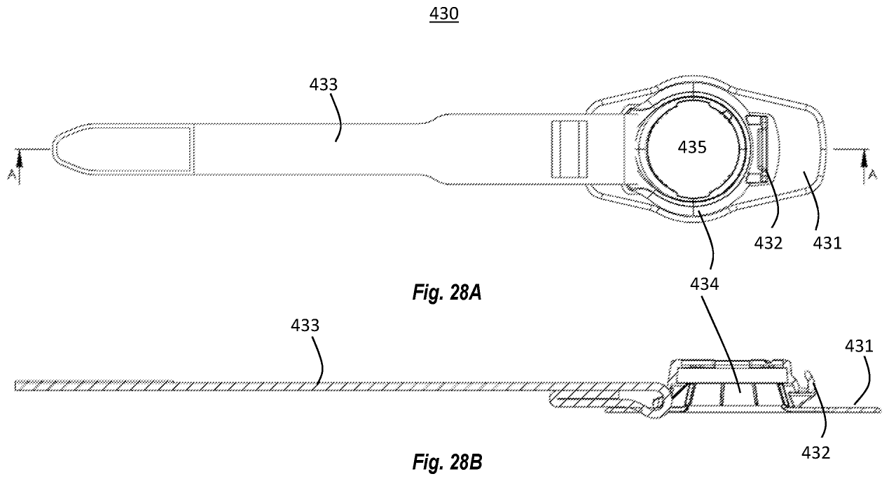

The fluid delivery apparatus 10 includes the attachment band 430 with an annular body 432 and a strap assembly 433 as shown in FIGS. 28A-29B. FIGS. 28A-28B show top and sectional views of an attachment band assembly, respectively according to an embodiment of the device described herein. FIG. 29A is a top view of an attachment ring of the attachment band assembly shown in FIGS. 28A-28B. FIG. 29B is a sectional view of the attachment ring of the attachment band assembly shown in FIGS. 28A-28B.

The attachment band 430 is configured to couple to the collet assembly 12 to facilitate attaching the fluid delivery apparatus 10 to a user during use. The band 430 includes an annular body having a wall 434 that is formed in a generally frustoconical shape, having a hollow inner space 435 defined therein. The annular body is sized and shaped to correspond to the upper wall 30 and the lower wall 34 of the collet 22. As illustrated in FIG. 29A, the inner wall of the attachment band 430 includes a plurality of tabs 436 (four sets of three tabs in FIG. 29A) that are configured to snap into the recessed portion 41 between the upper wall 30 and the lower wall 32 of the collet 22 (as shown in FIG. 4).

The attachment band 430 includes an inner step that extends circumferentially around an inner surface of the wall 434 of the annular body 432. In the exemplary embodiment, the inner step corresponds to the step 38 and the horizontal surface 40 that extends around the upper wall 30 of the collet 22.

In use, the attachment band can be stretched and tightened around the user's body part, such as an arm or wrist of the user. The band provides a generally axial force to the fluid delivery apparatus 10, generally along the central axis. The force of the fluid delivery apparatus 10 against the user's body facilitates causes the portion of the user's skin beneath the fluid delivery apparatus 10 to form a crown within the collet assembly 12. The collet assembly 12 also facilitates maintaining an appropriate amount of deformation (strain) of the user's skin during use of the fluid delivery apparatus 10. The skin deformation and the crowning of the portion of the user's skin encircled by the collet assembly 12 facilitate proper penetration of the protrusions of the fluidic distribution assembly 108 into the user's skin.

### H. Applicator

An applicator 500 (or broadly an application device) is optionally provided to facilitate the transition of the fluid delivery apparatus 10 from the non-activated configuration shown in FIGS. 2A and 3A to the activated configuration shown in FIGS. 2B and 3B. FIG. 30A is a perspective view of one suitable embodiment of the applicator 500 of the fluid delivery apparatus 10. FIG. 30B is a front sectional view of the applicator 500. FIG. 30C is a side sectional view of the applicator 500. In the exemplary embodiment, the applicator 500 has a housing 502 with a button 504 (or release) for activating the applicator 500. The housing 502 encloses a piston 506 (or impact component) used to activate the fluid delivery apparatus 10. The piston is locked into a safety position by one or more safety arms 508, 509. In addition, the housing encloses safety arm springs 510, piston spring 512, and button spring 514.

In the exemplary embodiment, the elongate body 520 has a generally cylindrical shape tapering inwardly from a bottom 516 to a top 518 of the body 520. The housing 502 also includes a cap 522 coupled to the top 518 of the body 520. The cap 522 is configured to retain the button 504, which is configured to move axially with respect to the body 520. It is noted that the applicator 500 is formed substantially symmetrical about an X-Y plane and a Y-Z plane that includes the centerline “E” as shown in FIG. 30A.

With reference to the FIGS. 30B-30C, the body 520 includes a stepped bore 528 that extends through the body 520. At the bottom end 516, the stepped bore 528 includes a first step portion 530 that has a periphery that is sized and shaped to receive the upper wall 30 of the collet 22 therein. As shown in FIG. 30B, the first step portion 530 extends upwardly from the bottom 516 of the body 520 a predetermined distance 532. The stepped bore 528 also includes a second step portion 534 that extends upwardly from the first step portion 530 a predetermined distance 536. In the exemplary embodiment, the second step portion 534 has a periphery that is sized and shaped to receive the fluid distribution assembly 14 while the first step portion 530 is in contact with the upper wall 30 of the collet 22. In addition, the stepped bore 528 includes a third step portion 538 that extends upwardly from the second step portion 534 and continues through the body 520. Positioned inside the body 520, and in particular, the third step portion 538 is a retaining ring 525. The retaining ring 525 is configured facilitate retaining the piston 506 and the safety arms 508, 509 axially within the housing 502. In addition, the third step portion 538 includes a plurality of axially-extending grooves 540 that extend upwardly from the second step portion 534 a predetermined distance 542. The grooves 540 have a curved cross-sectional shape that is generally centered on a radially extending line from the centerline “E.” That is, the grooves 540 extend axially through the second step portion 534 and are arranged radially about the centerline. Alternatively, the cross-sectional shape of the grooves 540 can be any shape that enables the applicator 500 to function as described herein. In the exemplary embodiment, the third step portion 538 has a periphery that is sized and shaped to receive the piston 506 therein.

In the exemplary embodiment, the third step portion 538 of the stepped bore 528 includes a piston retention member 546 that is positioned a predetermined distance 544 upwardly from the grooves 540. The piston retention member 546 is formed from a body that extends radially inwardly from an outer wall 548 of the body 520 and is configured to facilitate locking the piston 506 in place until the safety arms 508, 509 are actuated, thereby unlocking the piston 506. In addition, the piston retention member 546 functions as a spring seat for the piston spring 512 that is positioned between the piston 506 and the piston retention member 546, and the button spring 514 that is positioned between the button 504 and the piston retention member 546.

The body 520 also includes an opposing pair of longitudinal channels 550 that extend axially through the body 520. The channels 550 extend through the second and third step portions 534, 538, respectively, of the stepped bore 528. As best illustrated in FIG. 30B, the channels 550 are formed in the wall 548 of the body 520 and taper outward at the bottom 516 from the third step portion 538 to the second step portion 534. As such, the safety arms 508, 509 can be inserted into the channels 550 such that they do not interfere with the fluid delivery apparatus 10 during activation and/or use of the applicator 500. Thus, the channels 550 are sized and shaped to receive a respective safety arm 508, 509 slidingly therein, i.e., the safety arms 508, 509 are free to slide axially within the body 520 during use of the applicator 500.

### II. Methods of Using a Fluid Delivery Device

In some embodiments, a method for using the fluid delivery device described herein is provided. In some embodiments, a method for delivering a fluidic composition across a dermal barrier of a subject is provided. In some embodiments, the method comprises: inserting the plurality of protrusions of the device of any of the preceding claims across the dermal barrier of the subject; and transporting the fluidic composition through the fluidic path of the plurality of protrusions to a location below the dermal barrier.

In some embodiments, a method for delivering a fluidic composition across a dermal barrier of a subject is provided, the method comprising: penetrating the dermal barrier with a device having a plurality of protrusions with a nanopatterned layer comprising nanostructures overlaid thereon; and transporting the fluidic composition through the fluidic path of the plurality of protrusions to a location below the dermal barrier, wherein the number of protrusions in the plurality of protrusions is from about 100 to about 400 protrusions, and the fluidic composition is transported to a location below the dermal barrier at a flow rate greater than about 0.1 μl/hour per protrusion, or at a flow rate ranging from about 0.1 μl/hour to about 10 μl/hour per protrusion.

In some embodiments, the method further includes transporting the fluidic composition to the lymphatic system of the subject. In some embodiments, the method further includes transporting the fluidic composition to the blood circulatory system of the subject.

In some embodiments, the device may be placed in direct contact with the skin of the subject. In some embodiments, an intervening layer or structure may be placed between the skin of the subject and the medical device. For example, surgical tape or gauze may be used to reduce possible skin irritation between the device and the skin of the patient. When the protrusions extend from the apparatus, they will contact and, in some instances, penetrate the epidermis or dermis of the patient in order to deliver the medicament to the patient. The delivery of the fluidic composition can be to the blood circulatory system, the lymphatic system, the interstitium, subcutaneous, intramuscular, intradermal or a combination thereof. In some embodiments, the fluidic composition is delivered directly to the lymphatic system of the patient. In some embodiments, the fluidic composition is delivered to the superficial vessels of the lymphatic system.

In some embodiments, placement of the device proximate to the target results in the administered fluidic composition entering the lymphatic system and traversing to the intended target. The term “proximate” as used herein is intended to encompass placement on and/or near a desired target. In some embodiments, placement of the device may be such that the administered fluidic composition is directly administered to the target.

In some embodiments, the device is applied to an area of the subject's skin, in which a dense network of lymphatic capillaries and/or blood capillaries is present. Multiple devices may be applied to one or more locations within the area. In some embodiments, 1, 2, 3, 4, 5, or more devices may be applied. These devices may be applied spatially separate or in close proximity or juxtaposed with one another.

In some embodiments, at least a portion of or all of the fluidic composition may be directly delivered or administered to an initial depth in the skin comprising the nonviable epidermis and/or the viable epidermis. In some embodiments, a portion of the fluidic composition may also be directly delivered to the viable dermis in addition to the epidermis. The range of delivery depth will depend on the medical condition being treated and the skin physiology of a given subject. This initial depth of delivery may be defined as a location within the skin, wherein a therapeutic agent first comes into contact as described herein. Without being bound by any theory, it is thought that the administered agent may move (e.g., diffuse) from the initial site of delivery (e.g., the non-viable epidermis, the viable epidermis, the viable dermis, or the interstitium) to a deeper position within the viable skin. For example, a portion of or all of an administered agent may be delivered to the non-viable epidermis and then continue to move (e.g., diffuse) into the viable epidermis and past the basal layer of the viable epidermis and enter into the viable dermis. Alternatively, a portion of or all of an administered agent may be delivered to the viable epidermis (i.e., immediately below the stratum corneum) and then continue to move (e.g., diffuse) past the basal layer of the viable epidermis and enter into the viable dermis. Lastly, a portion of or all of an administered agent may be delivered to the viable dermis. The movement of the one or more active agents throughout the skin is multifactorial and, for example, depends on the liquid carrier composition (e.g., viscosity thereof), rate of administration, delivery structures, etc. This movement through the epidermis and into the dermis may be further defined as a transport phenomenon and quantified by mass transfer rate(s) and/or fluid mechanics (e.g., mass flow rate(s)).

Thus, in some embodiments described herein, the agent may be delivered to a depth in the epidermis wherein the agent moves past the basal layer of the viable epidermis and into the viable dermis. In some embodiments, the agent is then absorbed by one or more susceptible lymphatic capillary plexus then delivered to one or more lymph nodes and/or lymph vessels.

Because the thickness of the skin can vary from subject to subject based on numerous factors, including, but not limited to, medical condition, diet, gender, age, body mass index, and body part, the required depth to deliver the fluidic composition will vary. In some aspects, the delivery depth is from about 50 μm to about 4000 μm, from about 100 to about 3500 μm, from about 150 μm to about 3000 μm, from about 200 μm to about 3000 μm, from about 250 μm to about 2000 μm, from about 300 μm to about 1500 μm, or from about 350 μm to about 1000 μm. In some aspects, the delivery depth is about 50 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, or about 1000 μm. As used in this context, “about” means ±50 μm.

In some embodiments, the fluidic composition is delivered to the interstitium of the patient, e.g., to a space between the skin and one or more internal structures, such as an organ, muscle, or vessel (artery, vein, or lymph vessel), or any other spaces within or between tissues or parts of an organ. In some embodiments, the fluidic composition is delivered to both the interstitium and the lymphatic system.

### A. Fluidic Composition

In some embodiments, a fluidic composition comprises one or more agents (e.g., bioactive, diagnostic, or therapeutic agent or the like) in a liquid carrier solution.

In some embodiments, the fluidic composition has a viscosity from about 1 centipoise to about 100 centipoise. In some embodiments, the fluidic composition has a viscosity from about 1 centipoise to about 5 centipoise. In some embodiments, the fluidic composition has a viscosity of greater than about 5 centipoise. Any of the foregoing values may refer to viscosity at ambient temperature, e.g., 22° C. In some embodiments, the fluidic composition has the one or more agents at a concentration of greater than about 5 mg/mL. In some embodiments, the fluidic composition has the one or more agents at a concentration of from about 5 mg/mL to about 100 mg/mL.

In some embodiments, the tonicity of a liquid carrier solution may be hypotonic to the fluids within the blood capillaries or lymphatic capillaries. In another aspect, the tonicity of a liquid carrier solution may be isotonic to the fluids within the blood capillaries or lymphatic capillaries. The liquid carrier solution may further comprise at least one or more pharmaceutically acceptable excipients, diluent, cosolvent, particulates, or colloids. Pharmaceutically acceptable excipients for use in liquid carrier solutions are known, see, for example, Pharmaceutics: Basic Principles and Application to Pharmacy Practice (Alekha Dash et al. eds., 1st ed. 2013), which is incorporated by reference herein for its teachings thereof.

In some embodiments described herein, the agent is present in a liquid carrier as a substantially dissolved solution, a suspension, or a colloidal suspension. Any suitable liquid carrier solution may be utilized that meets at least the United States Pharmacopeia (USP) specifications, and the tonicity of such solutions may be modified as is known, see, for example, Remington: The Science and Practice of Pharmacy (Lloyd V. Allen Jr. ed., 22nd ed. 2012. Exemplary non-limiting liquid carrier solutions may be aqueous, semi-aqueous, or nonaqueous depending on the bioactive agent(s) being administered. For example, an aqueous liquid carrier may comprise water and any one of or a combination of a water-miscible vehicles, ethyl alcohol, liquid (low molecular weight) polyethylene glycol, and the like. Non-aqueous carriers may comprise a fixed oil, such as corn oil, cottonseed oil, peanut oil, or sesame oil, and the like. Suitable liquid carrier solutions may further comprise any one of a preservative, antioxidant, complexation enhancing agent, a buffering agent, an acidifying agent, saline, an electrolyte, a viscosity enhancing agent, a viscosity reducing agent, an alkalizing agent, an antimicrobial agent, an antifungal agent, a solubility enhancing agent or a combination thereof.

Non-limiting tests for assessing initial delivery depth in the skin may be invasive (e.g., a biopsy) or non-invasive (e.g., imaging). Conventional non-invasive optical methodologies may be used to assess delivery depth of an agent into the skin including remittance spectroscopy, fluorescence spectroscopy, photothermal spectroscopy, or optical coherence tomography (OCT). Imaging using methods may be conducted in real-time to assess the initial delivery depths. Alternatively, invasive skin biopsies may be taken immediately after administration of an agent, followed by standard histological and staining methodologies to determine delivery depth of an agent. For examples of optical imaging methods useful for determining skin penetration depth of administered agents, see, Sennhenn et al., Skin Pharmacol. 6(2) 152-160 (1993), Gotter et al., Skin Pharmacol. Physiol. 21 156-165 (2008), or Mogensen et al., Semin. Cutan. Med. Surg 28 196-202 (2009), each of which are incorporated by reference herein for their teachings thereof.

### B. Flow Rate

In some embodiments, a method for delivering a fluidic composition comprising one or more agents as described herein for a length of time, using the device described herein is provided. The length of time required may vary accordingly and accordingly the flow rate of the fluidic composition from the device into the subject can be adjusted. In some embodiments, the time period for administration is selected based on the medical condition of the subject and an assessment by the medical professional treating the subject. The flow rate will be based upon the medical condition of the subject and an assessment by the medical professional treating the subject.

In some embodiments, the flow rate is adjusted such that the fluidic composition is administered over from about 5 minutes to about 72 hours. In some aspects the time period for administration is about 5 minutes, 10 minutes, 15 minutes, 20 minutes, 0.5 hour, 1 hour, 2 hours, 3 hours, 4 hours, 5 hours, 6 hours, 7 hours, 8 hours, 9 hours, 10 hours, 12 hours, 15 hours 18 hours, 21 hours, 24 hours, 27 hours, 30 hours, 33 hours, 36 hours, 39 hours, 42 hours, 45 hours, 48 hours, 51 hours, 54 hours, 57 hours, 60 hours, 63 hours, 66 hours, 69 hours or 72 hours. In some embodiments, the time period for administration is in a range of 5 minutes to 10 minutes, 10 minutes to 15 minutes, 15 minutes to 20 minutes, 20 minutes to 0.5 hour, 0.5 hour to 1 hour, 1 hour to 2 hours, 2 hours to 3 hours, 3 hours to 4 hours, 4 hours to 5 hours, 5 hours to 6 hours, 6 hours to 7 hours, 7 hours to 8 hours, 8 hours to 9 hours, 9 hours to 10 hours, 10 hours to 12 hours, 12 hours to 15 hours, 15 hours to 18 hours, 18 hours to 21 hours, 21 hours to 24 hours, 24 hours to 27 hours, 27 hours to 30 hours, 30 hours to 33 hours, 33 hours to 36 hours, 36 hours to 39 hours, 39 hours to 42 hours, 42 hours to 45 hours, 45 hours to 48 hours, 48 hours to 51 hours, 51 hours to 54 hours, 54 hours to 57 hours, 57 hours to 60 hours, 60 hours to 63 hours, 63 hours to 66 hours, 66 hours to 69 hours, or 69 hours to 72 hours.

In some embodiments described herein, the flow rate of the fluidic composition per each protrusion as described herein may be greater than about 0.1 μl/hour. In some embodiments, the flow rate per protrusion is about 0.1 μl/hour to about 10 μl/hour. In some embodiments, the flow rate per protrusion is about 0.5 μl/hour to about 7.5 μl/hour. In some embodiments, the flow rate per protrusion is about 1 μl/hour to about 5 μl/hour. In some embodiments, the flow rate per protrusion is about 1.5 μl/hour to about 5 μl/hour. In some embodiments, the flow rate per protrusion is about 0.15 μl/hour to about 1.5 μl/hour. In some embodiments, the flow rate per protrusion is about 0.1 μl/hour, 0.15 μl/hour, 0.5 μl/hour, 1 μl/hour, 1.5 μl/hour, 2 μl/hour, 5 μl/hour, 7.5 μl/hour, or 10 μl/hour. In some embodiments, the flow rate per protrusion is about 0.5 μl/hour. In some embodiments, the flow rate per protrusion is about 1.5 μl/hour. In some embodiments, the flow rate is substantially uniform across substantially all of the protrusions. For example, the protrusion to protrusion variability of the flow rate may be less than 50%, less than 40%, less than 30%, less than 20%, or less than 10% over at least 75%, at least 85%, at least 90%, or at least 95% of the protrusions. In some embodiments, the protrusion to protrusion variability of the flow rate be about 10% or less. Each protrusion will have a flow rate that contributes to the overall device flow rate. The maximum overall flow rate will be a flow rate of each protrusion multiplied by the total number of protrusions.

The overall controlled flow rate of all of the combined protrusions (or the overall device flow rate) may be from about 0.4 μl/hour to about 25,000 μl/hour. In some embodiment, the overall device flow rate is from about 1 μl/hour to about 25,000 μl/hour, from about 10 μl/hour to about 20,000 μl/hour, from about 100 μl/hour to about 25,000 μl/hour, from about 200 μl/hour to about 15,000 μl/hour, from about 500 μl/hour to about 10,000 μl/hour, or from about 1000 μl/hour to about 5,000 μl/hour. In some aspects, the overall device flow rate is about 10 μl/hour, 100 μl/hour, 200 μl/hour, 500 μl/hour, 1000 μl/hour, 1,500 μl/hour, 2,000 μl/hour, 2,500 μl/hour, 3,000 μl/hour, 5,000 μl/hour, 10,000 μl/hour, or 20,000 μl/hour. In some embodiments, the overall device flow rate is about 100 μl/hour. In some embodiments, the overall device flow rate is about 500 μl/hour.

In some embodiments, the protrusions are arranged in 10 rows and 10 columns, and the device is capable of delivering the flow composition with the overall device flow rate of from about 10 μl/hour to 1,000 μl/hour. In some embodiments, the protrusions are arranged in 10 rows and 10 columns, and the device is capable of delivering the flow composition with the overall device flow rate of about 100 μl/hour. In some embodiments, the protrusions are arranged in 18 rows and 18 columns, and the device is capable of delivering the flow composition with the overall device flow rate of from about 32.4 μl/hour to 3,240 μl/hour. In some embodiments, the protrusions are arranged in 18 rows and 18 columns, and the device is capable of delivering the flow composition with the overall device flow rate of about 500 μl/hour. In some embodiments, the protrusions are arranged in 50 rows and 50 columns, and the device is capable of delivering the flow composition with the overall device flow rate of from about 250 μl/hour to 25,000 μl/hour.

The device is configured such that that the flow rate can be controlled appropriately. For example, where there is a larger number of protrusions, the flow rate per protrusion can be lower; where there is a smaller number of protrusions, the flow rate of protrusions can be higher.

In some embodiments, the flow rate does not change (i.e., is constant) for at least a predetermined time period. In some embodiments, the flow rate of the fluidic composition increases for a predetermined time period. In some embodiments, the flow rate decreases for at least a predetermined time period. In some embodiments, the flow rate changes over time in a sinusoidal, parabolic, triangular, or step-wise manner (i.e., a triangular, sinusoidal, parabolic, or step-wise flow rate profile).

In some embodiments described herein, the fluidic composition is administered to an initial approximate volume of space below the outer surface of the skin. The fluidic composition initially delivered to the skin (e.g., prior to any subsequent movement or diffusion) may be distributed within, or encompassed by an approximate three-dimensional volume of the skin. The one or more initially delivered agents may exhibit a Gaussian distribution of delivery depths and may also have a Gaussian distribution within a three-dimensional volume of the skin tissue.

In some embodiments, the method further comprises increasing permeability of the lymphatic vasculature wherein the nanostructures are in contact with, or proximate to, epithelial cells of the subject, thereby opening intercellular junctions between the epithelial cells and facilitating the flow of the fluidic composition during transport to the location below the dermal barrier.

In some embodiments described herein, the device as described herein functions as a permeability enhancer and may increase the delivery of the fluidic composition through the epidermis. This delivery may occur through modulating transcellular transport mechanisms (e.g., active or passive mechanisms) or through paracellular permeation. Without being bound by any theory, the nanostructures of the nanopatterned layer may increase the permeability of one or more layers of the viable epidermis, including the epidermal basement membrane by modifying cell/cell tight junctions allowing for paracellular or modifying cellular active transport pathways (e.g., transcellular transport) allowing for diffusion or movement and/or active transport of an administered agent through the viable epidermis and into the underlying viable dermis. This effect may be due to modulation of gene expression of the cell/cell tight junction proteins. As previously mentioned, tight junctions are found within the viable skin and in particular the viable epidermis. The opening of the tight junctions may provide a paracellular route for improved delivery of any agent, such as those that have previously been blocked from delivery through the skin.

Interaction between individual cells and structures of the nanotopography may increase the permeability of an epithelial tissue (e.g., the epidermis) and induce the passage of an agent through a barrier cell and encourage transcellular transport. For instance, interaction with keratinocytes of the viable epidermis may encourage the partitioning of an agent into the keratinocytes (e.g., transcellular transport), followed by diffusion through the cells and across the lipid bilayer again. In addition, interaction of the nanotopography structure and the corneocytes of the stratum corneum may induce changes within the barrier lipids or corneodesmosomes resulting in diffusion of the agent through the stratum corneum into the underlying viable epidermal layers. While an agent may cross a barrier according to paracellular and transcellular routes, the predominant transport path may vary depending upon the nature of the agent.

In some embodiments, the device may interact with one or more components of the epithelial tissue to increase porosity of the tissue making it susceptible to paracellular and/or transcellular transport mechanisms. Epithelial tissue is one of the primary tissue types of the body. Epithelial tissues that may be rendered more porous may include both simple and stratified epithelium, including both keratinized epithelium and transitional epithelium. In addition, epithelial tissue encompassed herein may include any cell types of an epithelial layer including, without limitation, keratinocytes, endothelial cells, lymphatic endothelial cells, squamous cells, columnar cells, cuboidal cells and pseudostratified cells. Any method for measuring porosity may be used including, but not limited to, any epithelial permeability assay. For example, a whole mount permeability assay may be used to measure epithelial (e.g., skin) porosity or barrier function in vivo see, for example, Indra and Leid., Methods Mol Biol. (763) 73-81, which is incorporated by reference herein for its teachings thereof.

In some embodiments, the structural changes induced by the presence of a nanotopography (the nanopatterned layer having a plurality of nanostructures) on a barrier cell are temporary and reversible, including reversible increase in the porosity of epithelial tissues by changing junctional stability and dynamics, which, without being bound by any theory, may result in a temporary increase in the paracellular and transcellular transport of an administered agent through the epidermis and into the viable dermis. Thus, in some aspects, the increase in permeability of the epidermis or an epithelial tissue elicited by the nanotopography, such as promotion of paracellular or transcellular diffusion or movement of one or more agents, returns to a normal physiological state that was present before contacting the epithelial tissue with a nanotopography following the removal of the nanotopography. In this way, the normal barrier function of the barrier cell(s) (e.g., epidermal cell(s)) is restored and no further diffusion or movement of molecules occurs beyond the normal physiological diffusion or movement of molecules within the tissue of a subject.

These reversible structural changes induced by the nanotopography may function to limit secondary skin infections, absorption of harmful toxins, and limit irritation of the dermis. Also, the progressive reversal of epidermal permeability from the top layer of the epidermis to the basal layer may promote the downward movement of one or more agents through the epidermis and into the dermis and prevent back flow or back diffusion of the one or more agents back into the epidermis.

### C. Methods of Delivery to Lymphatic System

In some embodiments, a method for administering a fluidic composition to the lymphatic system of a patient is provided, comprising applying the fluid delivery device described herein to deliver the fluidic composition to the lymphatic system. Delivery to the lymphatic system encompasses, e.g., delivery to a target in the lymphatic system or delivery through the lymphatic system to the systemic circulation or to a non-lymphatic target, which may be a solid tumor, circulating cells, an organ, a tissue, a joint, etc.

The delivery target may be e.g., a solid tumor, lymph nodes, or a specifically inflamed joint in a patient. The fluidic composition may comprise one or more agents to be delivered to such a therapeutic target. In some embodiments, the therapeutic target is a lymph node, a lymph vessel, an organ that is part of the lymphatic system or a combination thereof. In some embodiments, the therapeutic target is a lymph node. In some embodiments, the therapeutic target is a specific lymph node as described elsewhere herein.

In some embodiments, delivery of the therapeutic agent to the lymphatic system is delivery into the vessels of the lymphatic vasculature, the lymph nodes as described elsewhere herein, or both. In some embodiments, delivery is to the superficial lymph vessels. In yet another aspect, delivery is to one or more lymph nodes. The specific target for delivery will be based on the medical needs of the patient.

In some embodiments, the device is applied to an area of the subject's skin, in which a dense network of lymphatic capillaries and/or blood capillaries is present. Exemplary and non-limiting locations dense with lymphatics comprise the palmar surfaces of the hands, the scrotum, the plantar surfaces of the feet and the lower abdomen. The location of the device will be selected based on the medical condition of the patient and the assessment of a medical professional.

In the methods disclosed herein, two different exemplary modes for delivering a therapeutic agent to a patient are envisioned. In one mode, the target for the therapeutic agent is clearly identified, and the medical device comprising a plurality of protrusions is placed such that the medicament is administered to the lymphatic system of the patient such that it is carried by the lymph vessels directly to that target. The target may be, e.g., a solid tumor or a specifically inflamed joint in a patient. In this case, while some systemic exposure will occur, the administration is much more regionalized. In the second mode, the therapeutic target or exact location of the target may be unknown or less clearly defined, delivery of the therapeutic agent is into the lymphatic system of the patient, and the agent is intended to traverse the lymphatic system to either the right lymphatic duct or the thoracic duct. The therapeutic agent then enters the circulatory system of the patient leading to systemic exposure to the agent. For example, if a solid tumor has metastasized, the location of secondary sites for these cancer cells may not be known. Also, for some inflammatory medical conditions (e.g., Crohn's disease), an exact target for delivery of the therapeutic agent is not known. Although the therapeutic agent may traverse certain lymph nodes before reaching either of the draining ducts, the administration is considered to result in systemic exposure. As such, one skilled in the art can apply methods disclosed to provide targeted, regional administration of a therapeutic agent or more widespread systemic administration. A medical professional can determine which mode of administration is appropriate for an individual patient and place the medical device or devices accordingly.

In patients where more than one medical device is used to deliver the therapeutic agent to a plurality of locations on the body of a patient, the overall dose of the therapeutic agent at each location must be carefully adjusted such that the patient does not receive an overall unsafe combined dose of the agent. Being able to more selectively target specific locations in or on the body of a patient more precisely often means a lower dose is required at each specific location. In some embodiments, the dose administered to target one or more locations on the body of a patient is lower than a dose administered by other routes, including intravenous and subcutaneous administration.

Because the lymph fluid circulates throughout the body of a patient in a similar manner to blood in the circulatory system, any single position in the lymphatic vasculature can be upstream or downstream relative to another position. As used herein in reference to the lymphatic vasculature, the term “downstream” refers to a position in the lymphatic system closer (as the fluid travels through the vessels in a healthy patient) to either the right lymphatic duct or the thoracic duct relative to the reference position (e.g., a tumor or internal organ or a joint). As used herein, the term “upstream” refers to a position in the lymphatic system that is farther from the right lymphatic duct or the thoracic duct relative to the reference position. Because the direction of fluid flow in the lymphatic system can be impaired or reversed due to the medical condition of the patient, the terms “upstream” and “downstream” do not specifically refer to the direction of fluid flow in the patient undergoing medical treatment. They are positional terms based on their physical position relative to the draining ducts as described.

Because lymph nodes often occur in a group as opposed to being present as a single isolated node, the term “lymph node” as used herein can be singular or plural and refer to either a single isolated lymph node or a group of lymph nodes in a small physical location. For example, a reference to the inguinal lymph node or inguinal lymph nodes refers to the group of lymph nodes that are recognized by a person skilled in the art (i.e., a medical professional such as a doctor or a nurse) as a group of lymph nodes located in the hip/groin area or femoral triangle in a patient. It also refers to both the superficial and deep lymph nodes unless specifically stated otherwise. In some aspects, the lymph node is the sentinel lymph node for a specific solid cancer tumor.

In some embodiments, the lymph node is selected from the group consisting of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes, and combinations thereof.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, two or more different lymph nodes are selected. In some embodiments, three or more different lymph nodes are selected. The lymph nodes may be on either side of the body of the patient. In yet another embodiment, the lymph node is the inguinal lymph node. The inguinal lymph node may be the right inguinal lymph node, the left inguinal lymph node or both. In yet another embodiment, the lymph node is the axillary lymph node. The axillary lymph node may be the right axillary lymph node, the left axillary lymph node or both.

In some embodiments, the medicament is delivered to the interstitium of the patient, e.g., to a space between the skin and one or more internal structures, such as an organ, muscle, or vessel (artery, vein, or lymph vessel), or any other spaces within or between tissues or parts of an organ. In still yet another embodiment, the medicament is delivered to both the interstitium and the lymphatic system. In embodiments where the therapeutic agent is delivered to the interstitium of the patient, it may not be necessary to locate the lymph nodes or lymphatic vasculature of the patient before administering the therapeutic agent.

### D. Delivery to Multiple Regions of the Lymphatic System

One embodiment disclosed herein is a method for administering a therapeutic agent to the lymphatic system of a patient. The method generally comprises placing a first medical device comprising a plurality of protrusions on the skin of the patient at a first location proximate to a first position under the skin of the patient, wherein the first position is proximate to lymph vessels and/or lymph capillaries that drain into the right lymphatic duct, and wherein the protrusions of the first medical device have a surface comprising nanotopography; placing a second medical device comprising a plurality of protrusions on the skin of the patient at a second location proximate to a second position under the skin of the patient, wherein the second position is proximate to lymph vessels and/or lymph capillaries that drain into the thoracic duct, and wherein the protrusions of the second medical device have a surface comprising nanotopography; inserting the plurality of protrusions of the first medical device into the patient to a depth whereby at least the epidermis is penetrated and an end of at least one of the protrusions is proximate to the first position; inserting the plurality of protrusions of the second medical device into the patient to a depth whereby at least the epidermis is penetrated and an end of at least one of the protrusions is proximate to the second position; and administering via the protrusions of the first medical device a first dose of the therapeutic agent into the first position; administering via the protrusions of the second medical device a second dose of the therapeutic agent into a second position; wherein administering the doses cumulatively provides a therapeutically effective amount of the therapeutic agent.

In another aspect, disclosed herein is a method for administering a therapeutic agent to the lymphatic system of a patient. The method generally comprises placing a first medical device comprising a plurality of protrusions on the skin of the patient at a first location proximate to a first position under the skin of the patient, wherein the first position is proximate to lymph vessels and/or lymph capillaries that drain into the right lymphatic duct, and wherein the protrusions of the first medical device have a surface comprising nanotopography; placing a second medical device comprising a plurality of protrusions on the skin of the patient at a second location proximate to a second position under the skin of the patient, wherein the second position is proximate to lymph vessels and/or lymph capillaries that drain into the thoracic duct, and wherein the protrusions of the second medical device have a surface comprising nanotopography; inserting the plurality of protrusions of the first medical device into the patient to a depth whereby at least the epidermis is penetrated and an end of at least one of the protrusions is proximate to the first position; inserting the plurality of protrusions of the second medical device into the patient to a depth whereby at least the epidermis is penetrated and an end of at least one of the protrusions is proximate to the second position; administering via the protrusions of the first medical device a first therapeutically effective dose of the therapeutic agent into the first position; and administering via the protrusions of the second medical device a second therapeutically effective dose of the therapeutic agent into the second position; wherein a beginning time for administering the first dose and the second dose are different and separated by a period of time.

In some aspects disclosed herein, the first position and second position are reversed and the first position is proximate to lymph vessels and/or lymph capillaries that drain into the thoracic duct and the second position is proximate to lymph vessels and/or lymph capillaries that drain into the right lymphatic duct. As noted, one medical device drains into one of the two draining ducts in the lymphatic system while the other medical device drains into the other draining duct. This method is envisioned to administer at least a therapeutic agent to the lymphatic system of the patient such that different parts of the lymphatic system are exposed to the therapeutic agent. In some aspects, two or more medical devices are placed such that they drain into the same draining duct but they target different regions of the lymphatic system of the patient. For example, one device may be placed on the left arm of the patient and one device may be placed on the left leg of the patient. Although the therapeutic agent would ultimately drain through the same duct for site of administration, the therapeutic agent would traverse significantly different regions of the lymphatic system of the patient.

In some aspects, the first dose of the therapeutic agent and the second dose of the therapeutic agent are not therapeutically effective individually, but the combined amount of the doses is therapeutically effective. The first dose and the second dose can be administered sequentially or simultaneously. In some aspects, the first dose and the second dose are administered sequentially. In some aspects, the first dose and the second dose are administered simultaneously. In some aspects, administration of the two doses at least partially overlaps in time. This means that the administration of the two doses commences at different times, but the administration of the second dose begins before the administration of the first dose ends.

The location on the body of the patient is selected based on the medical condition of the patient and the knowledge of the medical professional supervising, directing and/or administering the treatment. For each medical device used with the methods disclosed herein, the location of the medical device on the body of the patient is selected independently of the other medical devices with the caveat that the objective of this method is to expose different parts of the lymphatic system to the therapeutic agent. In some aspects, each medical device is placed on a limb (i.e., arm or leg) of the patient. In order to achieve maximum exposure of the lymphatic system to the therapeutic agent, one device is placed on the right arm of the patient while the other device is place on the left leg of the patient. Alternatively, one device could be placed on the left arm of the patient while the other device is placed on the right leg of the patient. In yet another aspect, one medical device is placed on the right arm of the patient while the other medical device is placed on either the left arm or left leg of the patient. In yet another aspect, one medical device is placed on the left arm of the patient and the other medical device is placed on the right arm or right leg of the patient. A device on the arm of the patient may be located proximate to the wrist or hand of the patient while a device on the patient may be located proximate to the ankle or foot of the patient.

In still yet another aspect, the methods disclosed herein further comprise placing a third medical device comprising a plurality of protrusions on the skin of the patient at a third location proximate to a third position under the skin of the patient, wherein the third position is proximate to lymph vessels and/or lymph capillaries; inserting the plurality of protrusions of the third medical device into the patient to a depth whereby at least the epidermis is penetrated and an end of at least one of the protrusions is proximate to the third position; and administering via the third medical device a third dose of said therapeutic agent; and wherein the third location is different than the first location and the second location, and the third position is different that the first position and the second position.

In still yet another aspect, the methods disclosed herein further comprise placing a fourth medical device comprising a plurality of protrusions on the skin of the patient at a fourth location proximate to a fourth position under the skin of the patient, wherein the fourth position is proximate to lymph vessels and/or lymph capillaries; inserting the plurality of protrusions of the fourth medical device into the patient to a depth whereby at least the epidermis is penetrated and an end of at least one of the protrusions is proximate to the fourth position; and administering via the fourth medical device a fourth dose of said therapeutic agent; and wherein the first location, the second location, the third location, and the fourth location are on different limbs of the patient.

For any of the methods disclosed in, including those that use two medical devices, three medical devices, or four medical devices, in some aspects, each medical device is placed such that it initially drains into different lymph nodes, and wherein the draining lymph nodes are selected from the group of lymph nodes found in the hands, the feet, thighs (femoral lymph nodes), arms, legs, underarm (the axillary lymph nodes), the groin (the inguinal lymph nodes), the neck (the cervical lymph nodes), the chest (pectoral lymph nodes), the abdomen (the iliac lymph nodes), the popliteal lymph nodes, parasternal lymph nodes, lateral aortic lymph nodes, paraaortic lymph nodes, submental lymph nodes, parotid lymph nodes, submandibular lymph nodes, supraclavicular lymph nodes, intercostal lymph nodes, diaphragmatic lymph nodes, pancreatic lymph nodes, cisterna chyli, lumbar lymph nodes, sacral lymph nodes, obturator lymph nodes, mesenteric lymph nodes, mesocolic lymph nodes, mediastinal lymph nodes, gastric lymph nodes, hepatic lymph nodes, and splenic lymph nodes.

In one non-limiting example where three medical devices are used on a patient, the first device is placed on the right forearm of the patient which would then drain into the right axillary lymph nodes; the second device is placed on the left forearm of the patient which would then drain into the left axillary lymph nodes; and the third device is placed on the left thigh of the patient which would then drain into the left inguinal lymph nodes. In this instance the second and third devices would both drain into the thoracic duct but the initial draining lymph nodes are different.

In some aspects, the first dose of the therapeutic agent, the second dose of the therapeutic agent, and if present, the third dose of the therapeutic agent and the fourth dose of the therapeutic agent may each be administered to the patient sequentially or simultaneously. Doses may be combined such that the first and second dose are administered simultaneously while the third and fourth dose are administered together but sequentially relative to the first and second doses. In another aspect, the first and third dose and simultaneously administered while the second and fourth dose are administered simultaneous with each other and sequentially with the first and third dose. In yet another aspect, each dose is administered sequentially.

### E. Methods of Sequential Delivery of Multiple Doses

For any individual dose or combination of doses that are administered sequentially, there is a predetermined period of time between the beginning of each administration. That predetermined period of time may be 15 minutes, 30 minutes, 45 minutes, 1 hour, 2 hours, 3 hours, 4 hours, 5 hours, 6 hours, 7 hours, 8 hours, 12 hours, 16 hours, 20 hours, 24 hours, 36 hours, 48 hours, 60 hours, or 72 hours, or a range from and to any adjoining pair of the foregoing times. The predetermined period may be from about 15 minutes to about 72 hours or a time increment therebetween. Each period of time is selected independently of any other period of time and is based on the medical needs of the patient and the assessment of the medical professional administering, supervising or directing the treatment of the patient. Because the time that it takes to administer a dose of the therapeutic agent with the medical device is not zero, it is possible that the initiation of administering a subsequent dose of the therapeutic agent will be before the completion of the administration of the prior dose. For example, the administration of the second dose of the therapeutic agent may begin before the administration of the first dose of the therapeutic agent is complete. In yet another aspect, the predetermined period of time is based on the ending of one dose and the initiation of the next dose.

### F. Delivery to Blood Circulatory System

In some embodiment, disclosed herein is a method for increasing the bioavailability of a therapeutic agent in a patient, the method comprising placing at least one device described herein on the skin surface of the subject; and administering a therapeutic agent with the at least one medical device to the subject.

In some embodiments, the methods for delivering a therapeutic agent to a patient as described herein result in an equivalent blood serum absorption rate of one or more therapeutic agents described herein as compared to intravenous, subcutaneous, intramuscular, intradermal or parenteral delivery routes while retaining relatively higher rates of lymphatic delivery as described herein. Without being bound by any theory, the rate of delivery and increased bioavailability may be due to the lymphatic circulation of one or more agents through the thoracic duct or the right lymphatic duct and into the blood circulation. Standard highly accurate and precise methodologies for measuring blood serum concentration and therapeutic monitoring at desired time points may be used that are well known in the art, such as radioimmunoassays, high-performance liquid chromatography (HPLC), fluorescence polarization immunoassay (FPIA), enzyme immunoassay (EMIT) or enzyme-linked immunosorbant assays (ELISA). For calculating the absorption rate using the methods described above, the drug concentration at several time points should be measured starting immediately following administration and incrementally thereafter until a Cₘₐₓ value is established and the associated absorption rate calculated.

This written description uses examples to disclose the subject matter herein, and also to enable any person skilled in the art to practice the subject matter this disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A device for delivering a fluidic composition across a dermal barrier of a subject, the device comprising:
a. a fluid distribution assembly comprising:
i. a base;
ii. a plurality of protrusions defined on the base, wherein each of the protrusions has a tip and a height at a microscale with a fluidic path defined therein along the height from the base;
iii. a nanopatterned layer comprising a plurality of nanostructures and covering a surface of the plurality of protrusions;
iv. a gasket comprising a pressure-sensitive adhesive (PSA) layer;
v. a fluidic distribution manifold configured to be fluidically connected with the fluidic path of the protrusions and to controllably distribute the fluidic composition among the plurality of protrusions through the fluidic path;
b. a plenum assembly slidably coupled to the fluidic block and configured to hold the fluidic distribution assembly;
c. a collet assembly constituting the housing of the device and configured to contact a surface of the subject's skin sufficient for penetration of the plurality of protrusions into the surface of the subject's skin and across the dermal barrier; and
d. a controller assembly slidably coupled to the fluidic block and configured to control the flow of the fluidic composition during delivery of the fluidic composition through the plurality of protrusions;
wherein the number of protrusions in the plurality of protrusions is from about 4 to about 3000 protrusions and the device is capable of controllably delivering the fluidic composition to a location below the dermal barrier at a flow rate of greater than about 0.1 μl/hour per protrusion, or at a flow rate ranging from about 0.1 μl/hour to about 10 μl/hour per protrusion.

2. A device for delivering a fluidic composition across a dermal barrier of a subject, the device comprising:
a. a fluid distribution assembly comprising:
i. a base;
ii. a plurality of protrusions defined on the base, wherein each of the protrusions has a tip and a height at a microscale with a fluidic path defined therein along the height from the base;
iii. a nanopatterned layer comprising a plurality of nanostructures and covering a surface of the plurality of protrusions;
iv. a gasket comprising a pressure-sensitive adhesive (PSA) layer;
v. a fluidic distribution manifold configured to be fluidically connected with the fluidic path of the protrusions and to controllably distribute the fluidic composition among the plurality of protrusions through the fluidic path, the fluid distribution manifold comprising:
an inlet channel;
a plurality of supply channels and resistance channels, wherein each supply channel is connected to a respective resistance channel that facilitates an increase in the resistance to the flow of the fluid;
an outlet channel aligned with and fluidically connected to the fluidic path of the protrusions;
b. a plenum assembly slidably coupled to the fluidic block and configured to hold the fluidic distribution assembly;
c. a collet assembly constituting the housing of the device and configured to contact a surface of the subject's skin sufficient for penetration of the plurality of protrusions into the surface of the subject's skin and across the dermal barrier; and
d. a controller assembly slidably coupled to the fluidic block and configured to control the flow of the fluidic composition during delivery of the fluidic composition through the plurality of protrusions;
wherein the device is capable of delivering the fluidic composition at a flow rate greater than about 0.1 μl/hour per protrusion, or at a flow rate ranging from about 0.1 μl/hour to about 10 μl/hour per protrusion.

3. A device for delivering a fluidic composition across a dermal barrier of a subject, the device comprising:
a. a fluid distribution assembly comprising:
i. a base;
ii. a plurality of protrusions defined on the base, wherein each of the protrusions has a tip and a height at a microscale with a fluidic path defined therein along the height from the base;
iii. a nanopatterned layer comprising a plurality of nanostructures and covering a surface of the plurality of protrusions;
iv. a gasket comprising a pressure-sensitive adhesive (PSA) layer;
v. a fluidic distribution manifold configured to be fluidically connected with the fluidic path of the protrusions and to controllably distribute the fluidic composition among the plurality of protrusions through the fluidic path;
b. a plenum assembly slidably coupled to the fluidic block and configured to hold the fluidic distribution assembly;
c. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
d. a controller assembly slidably coupled to the fluidic block and configured to control the flow of the fluidic composition during delivery of the fluidic composition through the plurality of protrusions;
e. an attachment band assembly configured to couple to the collet assembly to facilitate contact with a surface of the subject's skin sufficient for penetration of the plurality of protrusions into the surface of the subject's skin and across the dermal barrier, wherein the attachment band assembly comprises:
an annular body comprising a wall defining a hollow inner space and a coupling member to engage with a corresponding coupling member of the collet, wherein the annular body is configured to attach to the collet of the collet assembly; and
a strap assembly removably engaged with the annular body and comprising a hoop-and-loop type fastening strap such that in use, the strap is threaded through a portion of the annular body and folded back to tighten the strap around the skin of the subject.

4. A device for delivering a fluidic composition across a dermal barrier of a subject, the device comprising:
a. a fluid distribution assembly comprising:
i. a base;
ii. a plurality of protrusions defined on the base, wherein each of the protrusions has a tip and a height at a microscale with a fluidic path defined therein along the height from the base;
iii. a nanopatterned layer comprising a plurality of nanostructures and covering a surface of the plurality of protrusions;
iv. a gasket comprising a pressure-sensitive adhesive (PSA) layer;
v. a fluidic block configured to be fluidically connected with the fluidic path of the protrusions and to controllably distribute the fluidic composition among the plurality of protrusions through the fluidic path;
b. a plenum assembly slidably coupled to the fluidic block and configured to hold the fluidic distribution assembly, comprising
a plenum having a center portion removably connected to a tubing system to provide a pressured flow of the fluidic composition from a reservoir for holding the fluidic composition located exterior of the device; and
a plenum cap assembly configured to facilitate gas extraction from the fluid;
c. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
d. an external infusion pump configured to control the flow of the fluidic composition during delivery of the fluidic composition through the plurality of protrusions;
wherein the device is capable of controllably delivering the fluidic composition to a location below the dermal barrier, at a flow rate greater than about 0.1 μl/hour per protrusion, or at a flow rate ranging from about 0.1 μl/hour to about 10 μl/hour per protrusion.

5. A device for delivering a fluidic composition across a dermal barrier of a subject, the device comprising:
a. a fluid distribution assembly comprising:
i. a base;
ii. a plurality of protrusions defined on the base, wherein each of the protrusions has a tip and a height at a microscale with a fluidic path defined therein along the height from the base;
iii. a nanopatterned layer comprising a plurality of nanostructures and covering a surface of the plurality of protrusions;
iv. a gasket comprising a pressure-sensitive adhesive (PSA) layer;
v. a fluidic block configured to be fluidically connected with the fluidic path of the protrusions and to controllably distribute the fluidic composition among the plurality of protrusions through the fluidic path;
b. a plenum assembly slidably coupled to the fluidic block and configured to hold the fluidic distribution assembly, comprising
a plenum component;
a cannula around a center axis coupled in fluid communication with the fluidic block; and
a plenum cap assembly configured to facilitate gas extraction from the fluid;
c. a cartridge assembly comprising a reservoir component includes an upper cavity and an opposing lower cavity coupled together in flow communication with the fluidic block via the cannula of the plenum assembly;
d. a collet assembly constituting the housing of the device and comprising a collet and a collet lock, wherein the collet assembly is configured to contact a surface of the subject's skin sufficient for penetration of the plurality of protrusions into the surface of the subject's skin and across the dermal barrier; and
e. a controller assembly slidably coupled to the fluidic block and configured to control the flow of the fluidic composition during delivery of the fluidic composition through the plurality of protrusions, comprising:
a plunger member positionable in a range from a first position proximal to the plenum, to a second position distant from the plenum; and
a bias assembly positioned between the plenum and the plunger member, the bias assembly being configured to apply a pressure to the plunger member, wherein the pressure applied to the plunger member by the biasing assembly is transmitted to the plenum and facilitates displacing the fluidic composition into the fluidic block.
wherein the device is capable of penetrating the dermal barrier of the subject and controllably delivering the fluidic composition to a location below the dermal barrier, at a flow rate greater than about 0.1 μl/hour per protrusion, or at a flow rate ranging from about 0.1 μl/hour to about 10 μl/hour per protrusion.

6. The device of any one of claims 1-5, wherein the device is capable of delivering the fluidic composition to a location below the dermal barrier from about 50 μm to about 4000 μm, from about 250 μm to about 2000 μm, or from about 350 μm to about 1000 μm in depth.

7. The device of any one of claims 1-6, wherein the device is capable of delivering the fluidic composition to a location below the dermal barrier and proximate to the lymphatic vasculature of the subject.

8. The device of any one of claims 1-7, wherein the dermal barrier comprises the stratum corneum of the subject.

9. The device of any one of claims 1-7, wherein the dermal barrier comprises a portion of the epidermis of the subject.

10. The device of any one of claims 1-7, wherein the dermal barrier comprises the entire thickness of epidermis of the subject.

11. The device of any one of claims 1-7, wherein the dermal barrier comprises at least a portion of the dermis of the subject.

12. The device of any one of claims 1-11, wherein the device is capable of delivering the fluidic composition having a viscosity from about 1 centipoise to about 100 centipoise.

13. The device of any one of claims 1-12, wherein the device is capable of delivering the fluidic composition having a viscosity from about 1 centipoise to about 5 centipoise.

14. The device of any one of claims 1-13, wherein the device is capable of delivering the fluidic composition having a bioactive (diagnostic or therapeutic) agent in a concentration of from about 5 mg/mL to about 100 mg/mL.

15. The device of any one of claims 1-14, wherein the plurality of protrusions comprises from about 4 to about 3,000 protrusions

16. The device of any one of claims 1-15, wherein the plurality of protrusions comprises from about 100 to about 2,500 protrusions

17. The device of any one of claims 1-16, wherein the plurality of protrusions comprises about 100 protrusions.

18. The device of any one of claims 1-17, wherein the plurality of protrusions comprises about 324 protrusions.

19. The device of any one of claims 1-18, wherein the device is capable of delivering the flow composition at the flow rate per protrusion ranging from about 0.1 μl/hour to about 10 μl/hour, about 0.5 μl/hour to about 7.5 μl/hour, about 1 μl/hour to about 5 μl/hour, 1.5 μl/hour to about 5 μl/hour, or about 0.15 μl/hour to about 1.5 μl/hour.

20. The device of any one of claims 1-19, wherein the device is capable of delivering the flow composition at the flow rate per protrusion is about 0.1 μl/hour, 0.15 μl/hour, 0.5 μl/hour, 1 μl/hour, 1.5 μl/hour, 2 μl/hour, 5 μl/hour, 7.5 μl/hour, or 10 μl/hour.

21. The device of any one of claims 1-20, wherein the device is capable of delivering the flow composition at the overall device flow rate ranging from about 1 μl/hour to about 25,000 μl/hour, from about 10 μl/hour to about 20,000 μl/hour, from about 100 μl/hour to about 25,000 μl/hour, from about 200 μl/hour to about 15,000 μl/hour, from about 500 μl/hour to about 10,000 μl/hour, or from about 1000 μl/hour to about 5,000 μl/hour.

22. The device of any one of claims 1-21, wherein the device is capable of delivering the flow composition at the overall device flow rate of about 10 μl/hour, 100 μl/hour, 200 μl/hour, 500 μl/hour, 1000 μl/hour, 1,500 μl/hour, 2,000 μl/hour, 2,500 μl/hour, 3,000 μl/hour, 5,000 μl/hour, 10,000 μl/hour, or 20,000 μl/hour.

23. The device of any one of claims 1-22, wherein the device is capable of delivering the flow composition at the overall device flow rate of 100 μl/hour.

24. The device of any one of claims 1-22, wherein the device is capable of delivering the flow composition at the overall device flow rate of 500 μl/hour.

25. The device of any one of claims 1-24, wherein the protrusions of the plurality are arranged in an approximately evenly spaced pattern.

26. The device of any one of claims 1-25, wherein the protrusions are arranged in 2-50 rows and 2-50 columns in an equidistant manner.

27. The device of any one of claims 1-26, wherein the protrusions are arranged in 10 rows and 10 columns, and the device is capable of delivering the flow composition with the overall device flow rate of about 100 μl/hour.

28. The device of any one of claims 1-26, wherein the protrusions are arranged in 18 rows and 18 columns, and the device is capable of delivering the flow composition with the overall device flow rate of about 500 μl/hour.

29. The device of any one of claims 1-28, wherein the flow rate does not change for at least a predetermined time period.

30. The device of any one of claims 1-29, wherein the flow rate of the fluidic composition increases or decreases for a predetermined time period.

31. The device of any one of claims 1-30, wherein the flow rate changes over time in a sinusoidal, parabolic, triangular, or step-wise manner.

32. The device of any one of claims 1-31, wherein each of the protrusions has the height ranging from 1 μm to 1 mm, about 200 to about 800 μm, between about 250 to about 750 μm, or between about 300 to about 600 μm.

33. The device of any one of claims 1-32, wherein the protrusions have a cross-sectional dimension perpendicular to the height, wherein an aspect ratio of the height to the cross-sectional dimension is greater than 2, 3 or 4.

34. The device of any one of claims 1-33, wherein the fluidic path in the protrusions has a length and a cross-sectional dimension perpendicular to the length, wherein an aspect ratio of the length to the cross-section dimension ranges from about 1 to about 50, about 5 to about 40, or about 10 to about 20 in average.

35. The device of any one of claims 1-34, wherein the cross-sectional dimension of the fluidic path ranges from about 1 μm to about 100 μm, about 5 μm to about 50 μm, or about 10 μm to about 30 μm.

36. The device of any one of claims 1-35, wherein the nanostructures comprise a height and a cross-sectional dimension, and at least a portion of the nanostructures have one or more of the following characteristics:
a) center-to-center spacing of from about 50 nanometers to about 1 micrometer;
b) a height of from about 10 nanometers to about 20 micrometers;
c) an aspect ratio of the height to the cross-sectional dimension from about 0.15 to about 30;
d) the plurality of nanostructures constitute a nanopattern having a fractal dimension of greater than about 1;
e) the surface of the protrusion comprising a plurality of nanostructures having an average surface roughness ranging from about 10 nm to about 200 nm; and/or
f) an effective compression modulus ranging from about 4 MPa to about 320 MPa.

37. The device of any one of claims 1-36, wherein the nanopatterned layer further comprises a plurality of additional nanostructures having a cross-sectional dimension less than the cross-sectional dimension of the nanostructures.

38. The device of any one of claims 1-37, wherein the nanopatterned layer comprises a polyether ether ketone (PEEK) film.

39. The device of any one of claims 1-38, comprising a cartridge assembly including a reservoir component having an upper cavity and an opposing lower cavity coupled together in flow communication with the fluidic block.

40. The device of any one of claims 1-39, comprising a reservoir for holding the fluidic composition located exterior of the device, and fluidically connected to the fluidic block.

41. The device of any one of claims 1-40, wherein the collet assembly comprises a collet lock coupled to a collet.

42. The device of any one of claims 1-41, wherein the collet lock is permanently coupled to the collet, optionally wherein the coupling is via a UV-curable adhesive.

43. The device of any one of claims 1-42, further comprising an attachment band assembly configured to couple to the collet assembly to facilitate contact with a surface of the subject's skin sufficient for penetration of the plurality of protrusions into the surface of the subject's skin and across the dermal barrier.

44. The device of claim 43, wherein the attachment band assembly comprises:
a. an annular body comprising a wall defining a hollow inner space and a coupling member to engage with a corresponding coupling member of the collet, wherein the annular body is configured to attach to the collet of the collet assembly;
b. a strap assembly removably engaged with the annular body and comprising a hoop-and-loop type fastening strap such that in use, the strap is threaded through a portion of the annular body and folded back to tighten the strap around the skin of the subject.

45. The device of any one of claims 1-44, wherein the controller assembly includes a plunger member positionable in a range from a first position proximal to the plenum, to a second position distant from the plenum; and a bias assembly positioned between the plenum and the plunger member, the bias assembly being configured to apply a pressure to the plunger member.

46. The device of claim 45, wherein the pressure applied to the plunger member by the biasing assembly is transmitted to the plenum and facilitates displacing the fluidic composition into the fluidic block.

47. The device of any one of claims 1-46, wherein the controller assembly includes an external infusion pump and a tubing system to provide a pressured flow of the fluidic composition from a reservoir for holding the fluidic composition located exterior of the device into the device and through the plenum to the fluidic block.

48. The device of claim 47, wherein the external infusion pump is a syringe pump, an elastomeric pump, or a peristaltic pump.

49. The device of claim 48, wherein the external infusion pump is portable.

50. The device of any one of claims 1-49, wherein the device is capable of delivering the flow composition with a protrusion to protrusion variability of a flow rate less than 50%, less than 40%, less than 30%, less than 20%, or less than 10% over at least 75% of the protrusions.

51. The device of any one of claims 1-50, wherein the device is capable of delivering the flow composition with a protrusion to protrusion variability of the flow rate being about 10% or less.

52. A method for delivering a fluidic composition across a dermal barrier of a subject comprising:
inserting the plurality of protrusions of at least one device of any of the preceding claims across the dermal barrier of the subject; and
transporting the fluidic composition through the fluidic path of the plurality of protrusions to a location below the dermal barrier.

53. The method of claim 52, wherein the fluidic composition is delivered at a flow rate greater than about 0.4 μl/hour, or at a flow rate ranging from about 0.4 μl/hour to about 25,000 μl/hour.

54. A method for delivering a fluidic composition across a dermal barrier of a subject comprising:
penetrating the dermal barrier with a device having a plurality of protrusions with a nanopatterned layer comprising nanostructures overlaid thereon; and
transporting the fluidic composition through the fluidic path of the plurality of protrusions to a location below the dermal barrier,
wherein the number of protrusions in the plurality of protrusions is from about 4 to about 2,500 protrusions, and the fluidic composition is transported to a location below the dermal barrier at a flow rate greater than about 0.1 μl/hour per protrusion, or at a flow rate ranging from about 0.1 μl/hour to about 10 μl/hour per protrusion.

55. The method of any one of claims 52-54, further comprising transporting the fluidic composition to lymphatic vasculature of the subject.

56. The method of any one of claims 52-55, comprising increasing permeability of the lymphatic vasculature wherein the nanostructures are in contact with, or proximate to, epithelial cells of the subject, thereby opening intercellular junctions between the epithelial cells and facilitating the flow of the fluidic composition during transport to the location below the dermal barrier.

57. A method for delivering a fluidic composition across a dermal barrier of a subject comprising:
applying more than one device of any of the preceding claims at two or more positions of a subject; and
transporting the fluidic composition through the fluidic path of the plurality of protrusions to a location below the dermal barrier.

58. A method for delivering a fluidic composition across a dermal barrier of a subject comprising:
placing a first device of any of the preceding claims on the skin of a subject at a first position proximate to a first location below the dermal barrier; and
placing a second device of any of the preceding claims on the skin of the subject at a second position proximate to a second location below the dermal barrier; and
in one or more steps, inserting the plurality of protrusions of the first device into the subject to a depth whereby an end of at least one of the protrusions is proximate to the first location, and inserting the plurality of protrusions of the second device into the subject to a depth whereby an end of at least one of the protrusions is proximate to the second location; and
in one or more steps, administering via the protrusions of the first device a first dose of the fluidic composition into the first location; and administering via the protrusions of the second device a second dose of the fluidic composition into the second location.

59. The method of claim 58, wherein administering the first dose and administering the second dose is simultaneous.

60. The method of claim 58, wherein administering the first dose and administering the second dose partially overlap in time.

61. The method of claim 58, wherein administering the first dose and administering the second dose is sequential.

62. The method of any one of claims 58-61, wherein the first and second devices are different devices.

63. The method of any one of claims 58-61, wherein the first and second devices are the same device.

64. The method of any one of claims 58-63, wherein administering the doses cumulatively provides a therapeutically effective dose.

65. The method of any one of claims 58-64, wherein the first location and the second location are on different limbs of the subject.

66. The method of any one of claims 58-65, wherein the first location and the second location are each independently proximate to the hands or the feet of the patient.

67. The method of any one of claims 58-66, wherein the administration step is conducted for at least 4, 6, 8, 10, 12, 16, 24, 36, 48 or 72 hours.

68. The method of any one of claims 58-67, wherein the device is placed at a location on the skin of the subject having lymphatic capillaries and/or vessels that deliver lymph directly into the lymphatic system in an inflammatory locus in the patient comprising lymph nodes, lymph capillaries, lymph vessel, lymph organs or any combination thereof.
